(19) United States
(12) Patent Application Publication
(10) Pub. No.: US 2008/0200403 A1
(43) Pub. Date: Aug. 21, 2008

(54) 9A-Carbamoyl and Thiocarbamoyl Azalides with Anti-Inflammatory Activity

(75) Inventors: Ognjen Culic, Zagreb (HR); Martina Bosnar, Zagreb (HR); Nikola Marjanovic, Zagreb (HR); Vesna Erakovic, Rijeka (HR); Dubravko Jelic, Samobor (HR); Donatella Verbanac, Njivice (HR)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(73) Assignee: GLAXOSMITHKLINE

(21) Appl. No.: 11/813,870

(22) PCT Filed: Jan. 13, 2006
(86) PCT No.: PCT/IB2006/001088

§ 371 Date: Apr. 9, 2008

Related U.S. Application Data
(60) Provisional application No. 60/644,425, filed on Jan. 14, 2005.

(51) Int. Cl. A61K 31/7052; C12N 5/06; A61P 29/00

(52) U.S. Cl. 514/29; 435/375

(57) ABSTRACT

The use of semi-synthetic 9a-carbamoyl or thiocarbamoyl azalides for the treatment and prevention of inflammatory diseases and conditions in humans and animals. More particularly, the invention relates to the use of 15-membered azalides having at the 9a-position carbamoyl or thiocarbamoyl group, and their pharmaceutically acceptable derivatives for the treatment and prevention of inflammatory diseases and conditions in humans and animals.

### FIELD OF THE INVENTION

The present invention relates to the use of semi-synthetic 9a-carbamoyl or thiocarbamoyl azalides for the treatment and prevention of inflammatory diseases and conditions. More particularly, the invention relates to the use of 15-membered azalides having at the 9a-position a carbamoyl or thiocarbamoyl group, and their pharmaceutically acceptable derivatives for the treatment and prevention of inflammatory diseases and conditions.

### TECHNICAL FIELD

The invention is directed to solving the technical problem of providing novel targeted anti-inflammatory agents. More specifically, the invention provides anti-inflammatory agents wherein the active substance is neither a steroid nor an NSAID. The compounds of the invention are responsive to this problem by virtue of their anti-inflammatory activity and their ability to accumulate in various immune cells recruited to the locus of inflammation.

### BACKGROUND OF THE INVENTION

Inflammation is the final common pathway of various insults, such as infection, trauma, and allergies to the human body. It is characterized by activation of the immune system with recruitment of inflammatory cells, production of pro-inflammatory cells and production of pro-inflammatory cytokines.

Most inflammatory diseases are characterized by abnormal accumulation of inflammatory cells including monocytes/macrophages, granulocytes, plasma cells, lymphocytes and platelets. Along with tissue endothelial cells and fibroblasts, these inflammatory cells release a complex array of lipids, growth factors, cytokines and destructive enzymes that cause local tissue damage.

One form of inflammatory response is neutrophilic inflammation which is characterized by infiltration of the inflamed tissue by neutrophil polymorphonuclear leucocytes (PMN), which are a major component of host defense. Tissue infection by extracellular bacteria represents the prototype of this inflammatory response. On the other hand, various non-infectious diseases are characterized by extravascular recruitment of neutrophils. This group of inflammatory diseases includes chronic obstructive pulmonary disease, adult respiratory distress syndrome, some types of immune-complex alveolitis, cystic fibrosis, bronchitis, bronchiectasis, emphysema, glomerulonephritis, rheumatoid arthritis, gouty arthritis, ulcerative colitis, certain dermatoses such as psoriasis and vasculitis. In these conditions neutrophils are thought to play a crucial role in the development of tissue injury which, when persistent, can lead to the irreversible destruction of the normal tissue architecture with consequent organ dysfunction. Tissue damage is primarily caused by the activation of neutrophils followed by their release of proteinases and increased production of oxygen species.

Chronic obstructive pulmonary disease (COPD) is described by the progressive development of airflow limitation that is not fully reversible (ATC, 1995). Most patients with COPD have three pathological conditions; bronchitis, emphysema and mucus plugging. This disease is characterized by a slowly progressive and irreversible decrease in forced expiratory volume in the first second of expiration (FEV₁), with relative preservation of forced vital capacity (FVC) (Barnes, N. Engl. J. Med. (2000), 343(4): 269-280). In both asthma and COPD there is significant, but distinct, remodeling of airways. Most of the airflow obstruction is due to two major components, alveolar destruction (emphysema) and small airways obstruction (chronic obstructive bronchitis). COPD is mainly characterized by profound mucus cell hyperplasia.

Cigarette smoking, air pollution and other environmental factors are major causes of the disease. The causal mechanism remains currently undefined but oxidant-antioxidant disturbances are strongly implicated in the development of the disease. COPD is a chronic inflammatory process that differs markedly from that seen in asthma, with different inflammatory cells, mediators, inflammatory effects, and responses to treatment (Keatings et al., Am. J Respir. Crit. Care Med. (1996), 153: 530-534). Neutrophil infiltration of the patient's lungs is a primary characteristic of COPD.

Elevated levels of proinflammatory cytokines like TNF-α, and especially chemokines like IL-8 and GRO-α play a very important role in pathogenesis of this disease. Platelet thromboxane synthesis is also enhanced in patients with COPD (Keatings et al., Am.J. Respir. Crit. Care Med. (1996), 153: 530-534; Stockley and Hill, Thorax (2000), 55(7): 629-630). Most of the tissue damage is caused by activation of neutrophils followed by their release of (metallo)proteinases, and increased production of oxygen species (Repine et al., Am. J. Respir. Crit. Care Med. (1997), 156: 341-357; Barnes, Chest (2000), 117(2 Suppl): 10S-14S).

Most therapeutic endeavour is directed towards the control of symptoms (Barnes, Trends Pharm. Sci. (1998), 19(10): 415-423; Barnes, Am. J. Respir. Crit. Care Med. (1999) 160: S72-S79; Hansel et al., Expert Opin. Investig. Drugs (2000) 9(1): 3-23). Symptoms usually equate with airflow limitation and bronchodilators are the conventional therapy of choice. Prevention and treatment of complications, prevention of deterioration and improved quality and length of life are also primary goals stated in the three key international guidelines for the management of COPD (Culpitt and Rogers, Exp. Opin. Pharmacother. (2000) 1(5): 1007-1020; Hay, Curr. Opin. Chem. Biol. (2000), 4: 412-419). Basically, most of the current therapeutic research has been focused on mediators involved in the recruitment and activation of neutrophils, or attenuation of consequences of their undesirable activation (Stockley et al., Chest (2000), 117(2 Suppl): 58S-62S).

In 1975, TNF-α was defined as an endotoxin-induced serum factor causing tumor necrosis in vitro and in vivo (Carswell E. A. et al. Proc. Natl. Acad. Sci. U.S.A. 1975, 72, 3666-3670). In addition to antitumor activity, TNF-α has several other biologic activities that are important in homeostasis as well as in pathophysiological conditions. The main sources of TNF-α are monocytes-macrophages, T-lymphocytes and mast cells.

The finding that anti-TNF-α antibodies (cA2) are effective in the treatment of patients suffering from rheumatoid arthritis (RA) (Elliot M. et al. Lancet 1994, 344, 1105-1110) intensified the interest to find new TNF-α inhibitors as possible potent medicaments for RA. Rheumatoid arthritis is an autoinmune chronic inflammatory disease characterized by irreversible pathological changes of the joints. In addition to RA, TNF-α antagonists are also applicable to several other pathological conditions and diseases such as spondylitis, osteoarthritis, gout and other arthritic conditions, sepsis, septic shock, toxic shock syndrome, atopic dermatitis, contact dermatitis, psoriasis, glomerulonephritis, lupus erythematosus, scleroderma, asthma, cachexia, chronic obstructive lung disease, congestive heart failure, insulin resistance, lung (pulmonary) fibrosis, multiple sclerosis, Crohn's disease, ulcerative colitis, viral infections and AIDS.

The interest of the scientific community has recently turned towards the immunomodulating and anti-inflammatory activities of the macrolide antibiotics (Journal of Antimicrobial Chemotherapy, 1988, 41, Suppl. B, 37-46).

An ideal immunomodulating agent should be able to suppress the deleterious effects of the inflammatory response, while leaving the protective immune responses intact.

Macrolide antibiotics accumulate preferentially within different cells of subjects, especially within phagocyte cells such as mononuclear peripheral blood cells, and peritoneal and alveolar macrophages. (Gladue, R. P. et al, Antimicrob. Agents Chemother. 1989, 33, 277-282; Olsen, K. M. et al, Antimicrob. Agents Chemother. 1996, 40, 2582-2585). Anti-inflammatory effects of some macrolides have been described in the literature. For example, the anti-inflammatory effect of erythromycin derivatives have been described in J. Antimicrob. Chemother. 1998, 41, 37-46 and WO Patent Application No. 00/42055. Taisho claims further anti-inflammatory erythromycin derivatives modified in positions 3, 9, 11 and 12 (EP 0775489 and EP 0771564). In the patent application WO 02/087596, there is a good description of the anti-inflammatory activity of azithromycin, a known antibacterial agent. Azithromycin derivatives lacking the sugar moieties cladinose and desosamine and having anti-inflammatory activity have been described (Pliva, U.S. Pat. No. 4,886,792). International patent applications WO 04/039821 and WO 04/013153 (Zambon Group) disclose macrolide and azalide derivatives lacking cladinose sugar that exhibit anti-inflammatory but not antibacterial activity.

Anti-inflammatory effects of some macrolides are also known from in vitro and in vivo studies in experimental animal models such as in zymosan-induced peritonitis in mice (J. Antimicrob. Chemother. 1992, 30, 339-348) and endotoxin-induced neutrophil accumulation in rat trachea (J. Immunol. 1997, 159, 3395-4005). The modulating effect of macrolides upon cytokines such as interleukin 8 (IL-8) (Am. J. Respir. Crit. Care. Med. 1997, 156, 266-271) and interleukin 5 (IL-5) (EP Pat. No. 0775489 and EP Pat. No. 771564) is known as well.

Macrolides have proved to be useful in the treatment of inflammatory pathologies such as panbronchiolitis (Thorax, 1997, 52, 915-918), bronchial asthma (Chest, 1991, 99 670-673), and azithromycin in particular has proved effective in improving lung function in patients with cystic fibrosis (The Lancet, 1998, 351, 420).

The administration of macrolides to asthmatics is accompanied by a reduction in hypersecretion and in bronchial hypersensitivity resulting from the macrolides' anti-oxidative and anti-inflammatory interaction with phagocytes and in particular with neutrophils (Inflammation, Vol. 20, No. 6, 1996).

### SUMMARY OF THE INVENTION

The present invention relates to 9a-carbamoyl or thiocarbamoyl substituted 15-membered azalides having anti-inflammatory activity. No compound representing the subject of the present invention has been described either as an anti-inflammatory substance or as an inhibitor of TNF-α, IL-1, IL-6, IL-8, IL-2 or IL-5; and/or inhibitor of excessive lymphocyte proliferation; and/or excessive granulocyte degranulation. Consequently, the use of such compounds to combat inflammatory states such as diseases, conditions or disorders has not been described or suggested.

The present invention is directed to methods and uses for treatment of inflammatory diseases, conditions or disorders, or of diseases conditions or disorders associated with excessive expression of TNF-α, IL-1, IL-6, IL-8, IL-2 or IL-5 and/or inhibitor of excessive lymphocyte proliferation; and/or excessive granulocyte degranulation of compounds represented by Formula (I)

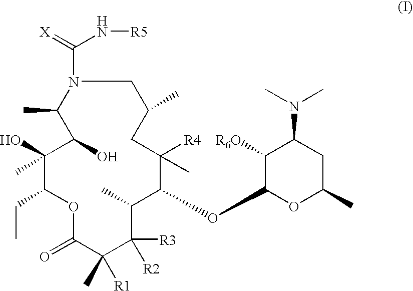

wherein
R¹ is hydrogen or together with R2 is a double bond;
R² is a cladinose sugar of formula (II), hydrogen, hydroxyl or a group of the formula (III) wherein Y is a monocyclic aromatic ring unsubstituted or substituted with a group selected from halogen, OH, OMe, NO₂, and NH₂; or

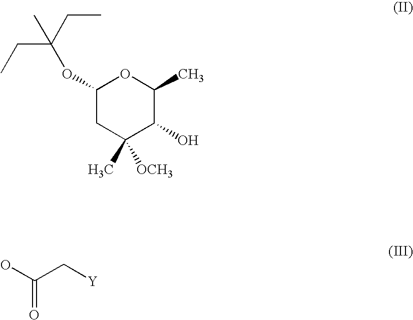

R² together with R³ is a ketone, or together with R¹ is a double bond;
R³ is hydrogen or together with R² is a ketone, or together with R⁴ represents an ether;
R⁴ is hydroxyl or OMe, or together with R³ is an ether;
R⁵ is C₁₋₄alkyl, C₂₋₄alkenyl, —(CH₂)ₘ—Ar, wherein Ar is a monocyclic or bicyclic aromatic ring up to 10 carbon atoms, containing 0-3 heteroatoms selected from N and O, unsubstituted or substituted by one or more of halogen, C₁₋₆haloalkyl, C₁₋₆haloalkoxy, C₁₋₆alkyl, C₁₋₆alkoxy, and m is 0-3,
R⁶ is hydrogen or a hydroxyl protecting group, and
X represents oxygen or sulfur; and
pharmaceutically acceptable derivatives thereof.

Moreover, no compound representing the subject of the present invention has been described either as an anti-inflammatory substance or as an inhibitor of one or more of TNF-α, IL-1, IL-6, IL-8, IL-2 or IL-5; and/or inhibitor of excessive lymphocyte proliferation; and/or excessive granulocyte degranulation.

### DETAILED DESCRIPTION OF THE INVENTION

In some embodiments, the methods of the present invention involve the treatment of inflammatory diseases, conditions or disorders, or of diseases conditions or disorders associated with excessive expression of TNF-α, IL-1, IL-6, IL-8, IL-2 or IL-5 and/or inhibition of excessive lymphocyte proliferation; and/or excessive granulocyte degranulation of compounds represented by Formula (I),

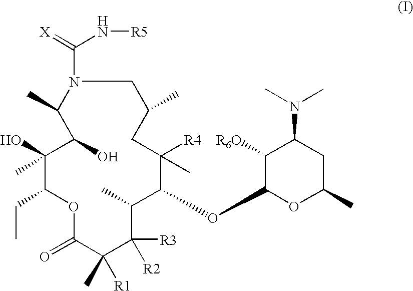

wherein
R¹ is hydrogen;
R² is a cladinose sugar of formula (II) or hydroxyl

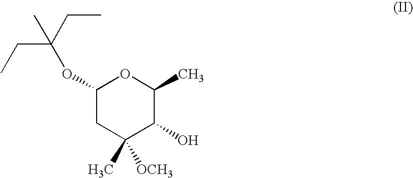

R³ is hydrogen or together with R⁴ represents an ether;
R⁴ is hydroxyl or together with R³ is an ether;
R⁵ is —(CH₂)ₘ—Ar, wherein Ar is a monocyclic or bicyclic aromatic ring up to 10 carbon atoms, unsubstituted or substituted by one or more of halogen, C₁₋₆haloalkyl, and m is 0-2,
R⁶ is hydrogen, and
X represents oxygen or sulfur; and
pharmaceutically acceptable derivatives thereof.

In one particular embodiment, the methods of the present invention involve the treatment of inflammatory diseases, conditions or disorders, or of diseases conditions or disorders associated with excessive expression of TNF-α, IL-1, IL-6, IL-8, IL-2 or IL-5 and/or inhibition of excessive lymphocyte proliferation; and/or excessive granulocyte degranulation of compounds represented by Formula (I), wherein
R¹ is hydrogen;
R² is a cladinose sugar of formula (II) or hydroxyl

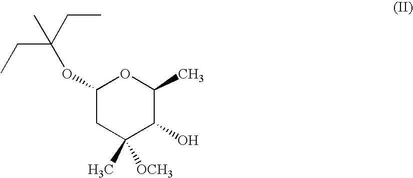

R³ is hydrogen or together with R⁴ represents an ether;
R⁴ is hydroxyl or together with R³ is an ether;
R⁵ is benzyl, 4-chlorophenyl, 3-fluorophenyl, 3-trifluoromethylphenyl, 2-fluorophenyl, 3-bromophenyl, 4-bromophenyl, 4-trifluoromethylphenyl, 3-trifluoromethyl-4-chlorophenyl, 1-(1-naphthyl)-ethyl,
R⁶ is hydrogen, and
X represents oxygen or sulfur; and
pharmaceutically acceptable derivatives thereof.

Particularly preferred compounds include:

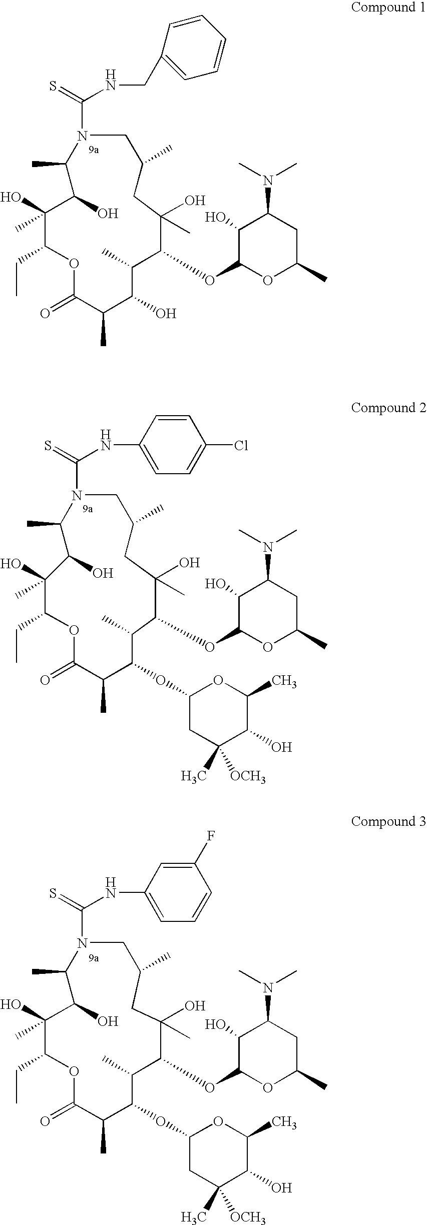

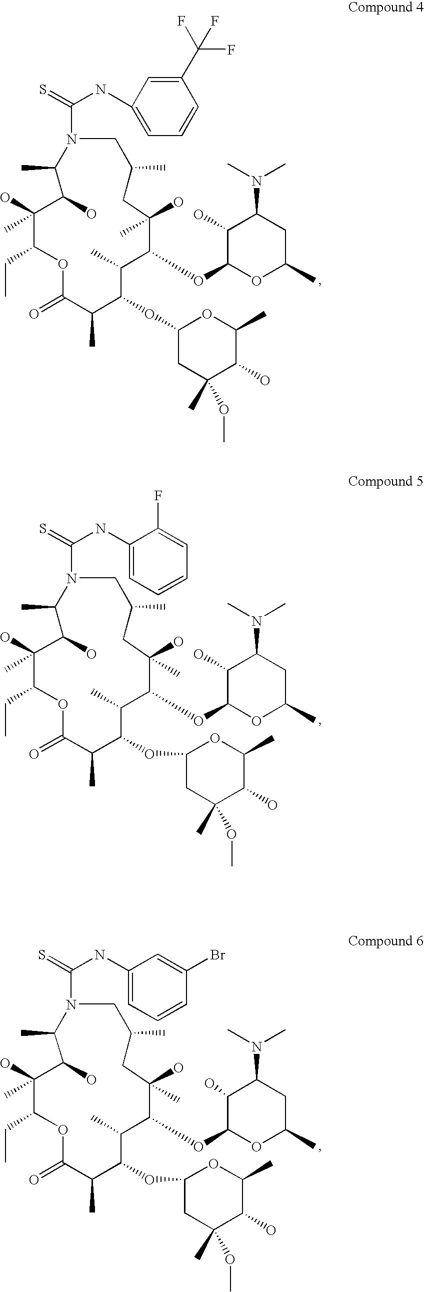

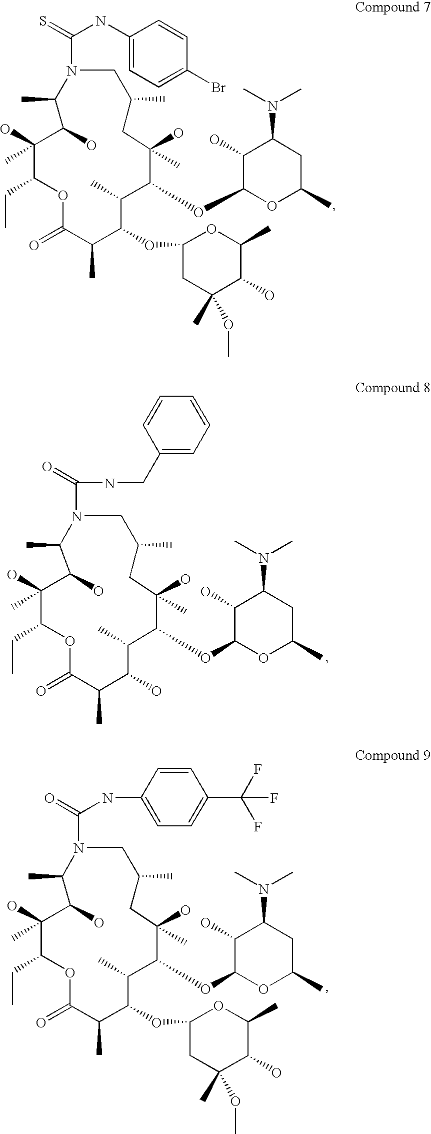

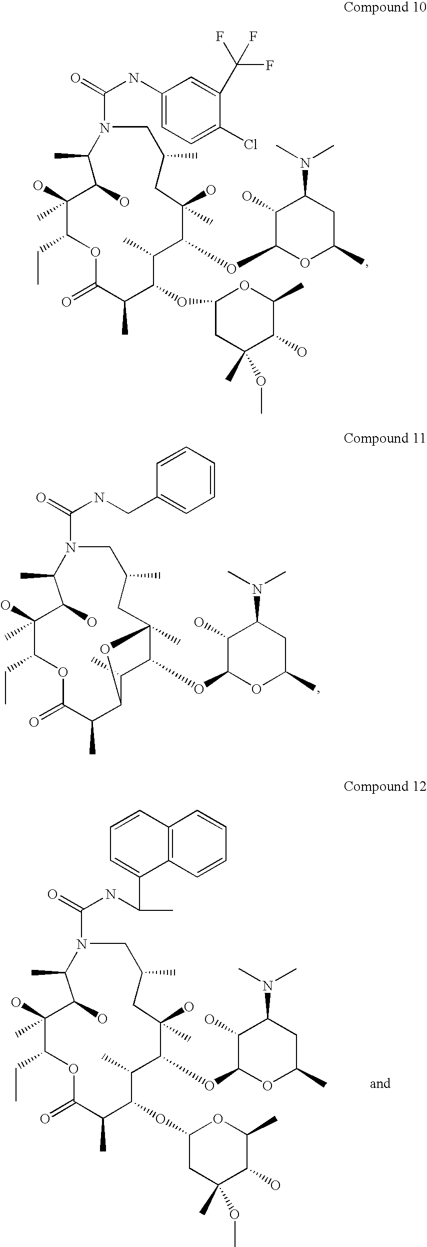

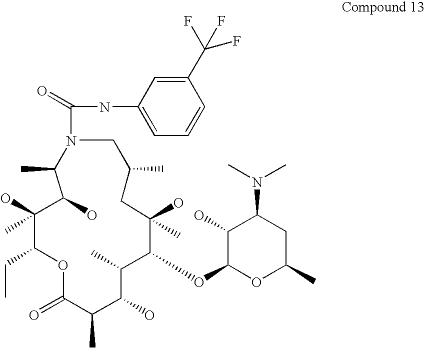

Azalide compounds described by Formula (I) may be prepared by methods described in U.S. Pat. No. 5,629,296, EP1175429, EP 0657464 and in international patent application WO 2004/101591, each disclosure of which is incorporated by reference herein in their entireties. Particularly, the compounds may be prepared in accordance with Examples 1-8 disclosed in U.S. Pat. No. 5,629,296 and processes analogous to these syntheses.

It will be appreciated by those skilled in the art that it may be desirable to use protected derivatives of intermediates used in the preparation of the compounds of formula (I). Protection and deprotection of functional groups may be performed by methods known in the art. Hydroxyl or amino groups may be protected with any hydroxyl or amino protecting group (for example, as described in Green and Wuts. Protective Groups in Organic Synthesis. John Wiley and Sons, New York, 1999). The protecting groups may be removed by conventional techniques. For example, acyl groups (such as alkanoyl, alkoxycarbonyl and aryloyl groups) may be removed by solvolysis (e.g., by hydrolysis under acidic or basic conditions). Arylmethoxycarbonyl groups (e.g., benzyloxycarbonyl) may be cleaved by hydrogenolysis in the presence of a catalyst such as palladium-on-carbon.

The synthesis of the target compound is completed by removing any protecting groups, which are present in the penultimate intermediate using standard techniques, which are well-known to those skilled in the art. The deprotected final product is then purified, as necessary, using standard techniques such as silica gel chromatography, HPLC on silica gel and the like, or by recrystallization.

It is therefore clear that the compounds of Formula (I), which have anti-inflammatory activity, can be useful in both acute and chronic treatment and in the prophylaxis of inflammatory pathologies, especially of those pathologies associated with altered cellular functionality of the neutrophils, for example and without limitation rheumatoid arthritis, vasculitis, glomerulonephritis, damage from ischemic reperfusion, atherosclerosis, septic shock, ARDS, COPD, cystic fibrosis and asthma.

The term “salts” can include acid addition salts or addition salts of free bases. Examples of acids which may be employed to form pharmaceutically acceptable acid addition salts include but are not limited to salts derived from nontoxic inorganic acids such as nitric, phosphoric, sulfuric, or hydrobromic, hydroiodic, hydrofluoric, phosphorous, as well as salts derived from nontoxic organic acids such as aliphatic mono- and dicarboxylic acids, phenyl-substituted alkanoic acids, hydroxyl alkanoic acids, alkanedioic acids, aromatic acids, aliphatic and aromatic sulfonic acids, and acetic, maleic, succinic, or citric acids. Non-limiting examples of such salts include napadisylate, besylate, sulfate, pyrosulfate, bisulfate, sulfite, bisulfite, nitrate, phosphate, monohydrogenphosphate, dihydrogenphosphate, metaphosphate, pyrophosphate, chloride, bromide, iodide, acetate, trifluoroacetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, maleate, tartrate, methanesulfonate, and the like. Also contemplated are salts of amino acids such as arginate and the like and gluconate, galacturonate (see, for example, Berge S. M. et al. “Pharmaceutical Salts,” J. of Pharma. Sci., 1977; 66:1).

The acid addition salts of said basic compounds are prepared by contacting the free base form with a sufficient amount of the desired acid to produce the salt in the conventional manner. The free base form may be regenerated by contacting the salt form with a base and isolating the free base in the conventional manner. The free base forms differ from their respective salt forms somewhat in certain physical properties such as solubility in polar solvents, but otherwise the salts are equivalent to their respective free base for purposes of the present invention.

Pharmaceutically acceptable base addition salts are formed with metals or amines, such as alkali and alkaline earth metals or organic amines. Examples of metals used as cations are sodium, potassium, magnesium, calcium, and the like. Examples of suitable amines are N,N′-dibenzylethylenediamine, chloroprocaine, choline, diethanolamine, dicyclohexylamine, ethylenediamine, N-methylglucamine, and procaine.

The base addition salts of said acidic compounds are prepared by contacting the free acid form with a sufficient amount of the desired base to produce the salt in the conventional manner. The free acid form may be regenerated by contacting the salt form with an acid and isolating the free acid.

The phrase “pharmaceutically acceptable”, as used in connection with compositions of the invention, refers to molecular entities and other ingredients of such compositions that are physiologically tolerable and do not typically produce untoward reactions when administered to a mammal (e.g., human). Preferably, as used herein, the term “pharmaceutically acceptable” means approved by a regulatory agency of the Federal or a state government or listed in the U.S. Pharmacopoeia or other generally recognized pharmacopeias for use in mammals, and more particularly in humans.

The term “pharmaceutically acceptable derivative” as used herein means any pharmaceutically acceptable salt, solvate or prodrug, e.g. ester, of a compound of the invention, which upon administration to the recipient is capable of providing (directly or indirectly) a compound of the invention, or an active metabolite or residue thereof. Such derivatives are recognizable to those skilled in the art, without undue experimentation. Nevertheless, reference is made to the teaching of Burger's Medicinal Chemistry and Drug Discovery, 5^{th} Edition, Vol 1: Principles and Practice, which is incorporated herein by reference to the extent of teaching such derivatives. Preferred pharmaceutically acceptable derivatives are salts, solvates, esters, carbamates and phosphate esters. Particularly preferred pharmaceutically acceptable derivatives are salts, solvates and esters. Most preferred pharmaceutically acceptable derivatives are salts and esters.

The compounds of Formula (I) may be administered with one or more carriers. The term “carrier” applied to pharmaceutical compositions of the invention refers to a diluent, excipient, or vehicle with which an active compound is administered. Such pharmaceutical carriers can be sterile liquids, such as water, saline solutions, aqueous dextrose solutions, aqueous glycerol solutions, and oils, including those of petroleum, animal, vegetable or synthetic origin, such as peanut oil, soybean oil, mineral oil, sesame oil and the like. Suitable pharmaceutical carriers are described in “Remington's Pharmaceutical Sciences” by E. W. Martin, 18th Edition. Particularly preferred for the present invention are carriers suitable for immediate-release, i.e., release of most or all of the active ingredient over a short period of time, such as 60 minutes or less, and make rapid absorption of the drug possible.

The present invention also encompasses prodrugs of the Formula (I) compounds, i.e., compounds which release an active parent drug according to Formula (I) in vivo when administered to a mammalian subject. Prodrugs of a compound of Formula (I) are prepared by modifying functional groups present in the compound of Formula (I) in such a way that the modifications may be cleaved in vivo to release the parent compound. Prodrugs include compounds of Formula (I) wherein a hydroxy, amino, or carboxy group of a Formula (I) compound is bonded to any group that may be cleaved in vivo to regenerate the free hydroxyl, amino or carboxy group, respectively. Examples of prodrugs include, but are not limited to esters (e.g., acetate, formate, and benzoate derivatives) of compounds of Formula (I) or any other derivative which upon being brought to the physiological pH or through enzyme action is converted to the active parent drug.

The present invention also encompasses solvates of the compounds of Formula (I) or their salts. Preferred solvates are hydrates.

The compounds of Formula (I) have one or more chirality centers and, depending on the nature of individual substituents, they can also have geometrical isomers. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has a chiral center, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomer respectively). A chiral compound can exist as either an individual enantiomer or as a mixture of enantiomers. A mixture containing equal proportions of the enantiomers is called a “racemic mixture”. The present invention encompasses all individual isomers of compounds of Formula (I). The description or naming of a particular compound in the specification and claims is intended to include both individual enantiomers and mixtures, racemic or otherwise, (i.e., enriched in one or more isomers) thereof. Methods for the determination of stereochemistry and the resolution of stereoisomers are well-known in the art.

The present invention also encompasses stereoisomers of the syn-anti type, and mixtures thereof encountered when an oxime or similar group is present. The group of highest Cahn Ingold Prelog priority attached to one of the terminal doubly bonded atoms of the oxime, is compared with hydroxyl group of the oxime. The stereoisomer is designated as Z (zusammen=together) or Syn if the oxime hydroxyl lies on the same side of a reference plane passing through the C═N double bond as the group of highest priority; the other stereoisomer is designated as E (entgegen=opposite) or Anti.

The term “alkyl” as used herein as a group or a part of a group refers to a straight or branched hydrocarbon chain containing the specified number of carbon atoms. For example, C₁₋₆ alkyl means a straight or branched alkyl chain containing from 1 to 6 carbon atoms; examples of such group include methyl, ethyl, propyl, isopropyl, n-butyl, isobutyl, tert-butyl, pentyl, 3-methyl-butyl, hexyl and 2,3-dimethylbutyl and like. Similarly, the term C1-4 alkyl means a straight or branched alkyl chain containing from 1 to 4 carbon atoms.

The term “alkenyl” as used herein as a group or a part of a group refers to a straight or branched hydrocarbon chain containing the specified number of carbon atoms and containing at least one double bond. For example, the term “C₂₋₄ alkenyl” means a straight or branched alkenyl containing at least 2, and at most 4, carbon atoms and containing at least one double bond. Examples of “alkenyl” as used herein include, but are not limited to, ethenyl, 2-propenyl, 3-butenyl, 2-butenyl, 2-methyl-2-propenyl, 2-methylbut-2-ethenyl. It will be appreciated that in groups of the form —O—C₂₋₆ alkenyl, the double bond is preferably not adjacent to the oxygen.

The term “alkoxy”, as used herein, refers to a straight or branched chain C₁₋₅ alkyl group, as previously defined, attached to the parent molecular moiety through an oxygen atom containing the specified number of carbon atoms. For example, C₁₋₄ alkoxy means a straight or branched alkoxy containing at least 1, and at most 4, carbon atoms. Examples of “alkoxy” as used herein include, but are not limited to, methoxy, ethoxy, propoxy, prop-2-oxy, butoxy, but-2-oxy, 2-methylprop-1-oxy and 2-methylprop-2-oxy.

The terms “haloalkyl” and “haloalkoxy” refers “alkyl” and “alkoxy” groups as defined above substituted with one are more halogen, where the halogen is a fluorine, chlorine, bromine or iodine atom.

The term “aryl”, as used herein, refers to a mono-, bicyclic carbocyclic ring system having one or two aromatic rings including, but not limited to, phenyl, naphthyl, tetrahydronaphthyl, indanyl, indenyl, and the like.

Depending on the type of formulation, in addition to a therapeutically effective quantity of one or more compounds of Formula (I), they will contain solid or liquid excipients or diluents for pharmaceutical use and possibly other additives normally used in the preparation of pharmaceutical formulations, such as thickeners, aggregating agents, lubricants, disintegrating agents, flavorings and colorants.

A “pharmaceutically acceptable excipient” means an excipient that is useful in preparing a pharmaceutical composition that is generally safe, non-toxic and neither biologically nor otherwise undesirable, and includes an excipient that is acceptable for veterinary use as well as human pharmaceutical use. A “pharmaceutically acceptable excipient” as used in the present application includes both one and more than one such excipient.

“Treating” or “treatment” of a state, disorder or condition includes:
(1) preventing or delaying the appearance of clinical symptoms of the state, disorder or condition developing in a mammal that may be afflicted with or predisposed to the state, disorder or condition but does not yet experience or display clinical or subclinical symptoms of the state, disorder or condition,
(2) inhibiting the state, disorder or condition, i.e., arresting, reducing or delaying the development of the disease or a relapse thereof (in case of maintenance treatment) or at least one clinical or subclinical symptom thereof, or
(3) relieving the disease, i.e., causing regression of the state, disorder or condition or at least one of its clinical or subclinical symptoms.

The benefit to a subject to be treated is either statistically significant or at least perceptible to the patient or to the physician.

A “therapeutically effective amount” means the amount of a compound that, when administered to a mammal for treating a state, disorder or condition, is sufficient to effect such treatment. The “therapeutically effective amount” will vary depending on the compound, the disease and its severity and the age, weight, physical condition and responsiveness of the mammal to be treated.

The four classic symptoms of acute inflammation are redness, elevated temperature, swelling and pain in the affected area, and impairment or loss of function of the affected organ.

Symptoms and signs of inflammation associated with specific conditions include:
rheumatoid arthritis—pain, swelling, warmth and tenderness of the involved joints; generalized and morning stiffness;
insulin-dependent diabetes mellitus—insulitis; this condition can lead to a variety of complications with an inflammatory component, including: retinopathy, neuropathy, nephropathy; coronary artery disease, peripheral vascular disease, and cerebrovascular disease;
autoimmune thyroiditis—weakness, constipation, shortness of breath, puffiness of the face, hands and feet, peripheral edema, bradycardia;
multiple sclerosis—spasticity, blurry vision, vertigo, limb weakness, paresthesias;
uveoretinitis—decreased night vision, loss of peripheral vision;
lupus erythematosus—joint pain, rash, photosensitivity, fever, muscle pain, puffiness of the hands and feet, abnormal urinalysis (hematuria, cylinduria, proteinuria), glomerulonephritis, cognitive dysfunction, vessel thrombosis, pericarditis;
sclerodenna—Raynaud's disease; swelling of the hands, arms, legs and face; skin thickening; pain, swelling and stiffness of the fingers and knees, gastrointestinal dysfunction, restrictive lung disease; pericarditis,; renal failure;
other arthritic conditions having an inflammatory component such as rheumatoid spondylitis, osteoarthritis, septic arthritis and polyarthritis—fever, pain, swelling, tenderness;
other inflammatory brain disorders, such as meningitis, Alzheimer's disease, AIDS dementia encephalitis—photophobia, cognitive dysfunction, memory loss;
other inflammatory eye inflammations, such as retinitis—decreased visual acuity;
inflammatory skin disorders, such as, eczema, other dermatites (e.g., atopic, contact), psoriasis, bums induced by UV radiation (sun rays and similar UV sources)—erythema, pain, scaling, swelling, tenderness;
inflammatory bowel disease, such as Crolin's disease, ulcerative colitis—pain, diarrhea, constipation, rectal bleeding, fever, arthritis;
asthma—shortness of breath, wheezing;
other allergy disorders, such as allergic rhinitis—sneezing, itching, runny nose
conditions associated with acute trauma such as cerebral injury following stroke—sensory loss, motor loss, cognitive loss;
heart tissue injury due to myocardial ischemia—pain, shortness of breath;
lung injury such as that which occurs in adult respiratory distress syndrome—shortness of breath, hyperventilation, decreased oxygenation, pulmonary infiltrates;
inflammation accompanying infection, such as sepsis, septic shock, toxic shock syndrome—fever, respiratory failure, tachycardia, hypotension, leukocytosis;
other inflammatory conditions associated with particular organs or tissues, such as:
nephritis (e.g., glomerulonephritis)-oliguria, abnormal urinalysis;
inflamed appendix—fever, pain, tenderness, leukocytosis;
gout—pain, tenderness, swelling and erythema of the involved joint, elevated serum and/or urinary uric acid;
inflamed gall bladder—abdominal pain and tenderness, fever, nausea, leukocytosis;
shortness of breath, wheezing;
congestive heart failure—shortness of breath, rates, peripheral edema;
Type II diabetes—end organ complications including cardiovascular, ocular, renal, and peripheral vascular disease
lung (pulmonary) fibrosis—hyperventilation, shortness of breath, decreased oxygenation;
vascular disease, such as atherosclerosis and restenosis—pain, loss of sensation, diminished pulses, loss of function; and
alloimmunity leading to transplant rejection—pain, tenderness, fever.

Subclinical symptoms include without limitation diagnostic markers for inflammation the appearance of which may precede the manifestation of clinical symptoms. One class of subclinical symptoms is immunological symptoms, such as the invasion or accumulation in an organ or tissue of proinflammatory lymphoid cells or the presence locally or peripherally of activated pro-inflammatory lymphoid cells recognizing a pathogen or an antigen specific to the organ or tissue. Activation of lymphoid cells can be measured by techniques known in the art; for example by measuring one or more inflammatory cytokines or mediators.

Responsiveness of a subject to a treatment is assessed by whether a selected drug used in the acute phase causes the reduction of one or more clinical signs and symptoms described below.

In the context of the present invention, “preventing” is used primarily with reference to maintenance therapy for the prevention of recurrence of a symptom for the disease or any measure of inflammation such as a marker for inflammation. For example, prevention can be demonstrated in animals that spontaneously develop inflammatory bowel disease (IBD) (e.g. IL-10 deficient mice, TNF ΔARE or SAMP1/Yit mice) and includes the avoidance or the delay of occurrence of disease in treated animals (compared to untreated controls).

An example of “relieving” a subclinical symptom is the observation in a treated individual of abatement in the number of immune cells that secrete pro inflammatory cytokines or lymphokines or a decrease in the mRNA encoding such lymphokines or cytokines.

“Maintenance therapy” is therapy during a phase of the disease, disorder or condition following the achievement of remission (total or partial) of one or more symptoms of a disease until the next flare-up of the disease. Partial remission is the disappearance or alleviation of one or more of the symptoms normally associated with the disease state. By way of nonlimiting example and with particular reference to IBD, the hallmark of the acute phase include symptoms like nausea, diarrhea, vomiting, fever, abdominal tenderness, pain, cramps, in some cases anemia and malnutrition signs. Anal fistulas can appear. Stools can be bloody or occult bleeding can occur and be determined on assay. White blood cells are moderately elevated; sedimentation rate is often elevated and can be used to monitor the transition from active to remission phase. Hypokalemia, hypoalbuminemia, and hypocalcaemia can occur during acute phase. X-ray examination of abdomen and barium enema are used to find lesions in mucosa and inflamed tissue; CT scans and ultrasound can be used for the same purpose. Maintenance therapy starts in the moment when abnormal symptoms previously determined to be present return to normal values. Acute phase therapy usually lasts from 2 to 6 weeks, depending on the patient, and the therapy used. Length of maintenance treatment comprising administration of the compounds of the present invention during maintenance phase typically lasts from the induction of remission until the appearance of the disease flare-up or indefinitely if disease is controlled. Perhaps it could be possible to discontinue the administration of the compounds of the present invention after several years of adequate disease control but signs of disease reappearance should carefully be observed.

“Responder” refers to a patient that has previously responded to a treatment for an inflammatory disease disorder or condition involving administration of a particular active agents (or combination of active agents) in particular amount or amounts.

“Subject” refers to an animal, which is preferably a mammal and more preferably human or a domestic animal. Most preferably, the subject is a human. As used herein, the term patient is used synonymously with human (or mammalian) subject.

A “therapeutically effective amount” means the amount of a compound that, when administered to a mammal for treating a state, disorder or condition, is sufficient to effect such treatment. The “therapeutically effective amount” will vary depending on the compound, the disease and its severity and the age, weight, physical condition and responsiveness of the mammal to be treated.

### Pharmaceutical Compositions

While it is possible that, for use in the methods of the invention, a compound 25 of formula (I) may be administered as the bulk substance, it is preferable to present the active ingredient in a pharmaceutical formulation, e.g., wherein the agent is in admixture with a pharmaceutically acceptable carrier selected with regard to the intended route of administration and standard pharmaceutical practice.

The phrase “pharmaceutically acceptable” refers to molecular entities and compositions that are generally regarded as safe. In particular, pharmaceutically acceptable carriers used in the pharmaceutical compositions of this invention are physiologically tolerable and do not typically produce an allergic or similar untoward reaction (for example, gastric upset, dizziness and the like) when administered to a patient. Preferably, as used herein, the term “pharmaceutically acceptable” means approved by a regulatory agency of the Federal or a state government or listed in the U.S. Pharmacopoeia or other generally recognized pharmacopoeia for use in animals, and more particularly in humans.

A “pharmaceutically acceptable excipient” means an excipient that is useful in preparing a pharmaceutical composition that is generally safe, non-toxic and neither biologically nor otherwise undesirable, and includes an excipient that is acceptable for veterinary use as well as human pharmaceutical use. A “pharmaceutically acceptable excipient” as used in the present application includes both one and more than one such excipient.

The term “carrier” refers to a diluent, excipient, and/or vehicle with which an active compound is administered. The pharmaceutical compositions of the invention may contain combinations of more than one carrier. Such pharmaceutical carriers can be sterile liquids, such as water, saline solutions, aqueous dextrose solutions, aqueous glycerol solutions, and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin, 18th Edition.

The compounds of the invention may be formulated for administration in any convenient way for use in human or veterinary medicine and the invention therefore includes within its scope pharmaceutical compositions comprising a compound of the invention adapted for use in human or veterinary medicine. Such compositions may be presented for use in a conventional manner with the aid of one or more suitable carriers. Acceptable carriers for therapeutic use are well-known in the pharmaceutical art, and are described, for example, in Remington's Pharmaceutical Sciences, Mack Publishing Co. (A. R. Gennaro edit. 1985). The choice of pharmaceutical carrier can be selected with regard to the intended route of administration and standard pharmaceutical practice. The pharmaceutical compositions may comprise as, in addition to, the carrier any suitable binder(s), lubricant(s), suspending agent(s), coating agent(s), and/or solubilizing agent(s).

It will be appreciated that pharmaceutical compositions for use in accordance with the present invention may be in the form of oral, parenteral, transdermal, inhalation, sublingual, topical, implant, nasal, or enterally administered (or other mucosally administered) suspensions, capsules or tablets, which may be formulated in conventional manner using one or more pharmaceutically acceptable carriers or excipients.

There may be different composition/formulation requirements depending on the different delivery systems. It is to be understood that not all of the compounds need to be administered by the same route. Likewise, if the composition comprises more than one active component, then those components may be administered by different routes. By way of example, the pharmaceutical composition of the present invention may be formulated to be delivered using a mini-pump or by a mucosal route, for example, as a nasal spray or aerosol for inhalation or ingestible solution, or parenterally in which the composition is formulated by an injectable form, for delivery, by, for example, an intravenous, intramuscular or subcutaneous route. Alternatively, the formulation may be designed to be delivered by multiple routes.

The present invention further relates to pharmaceutical formulations containing a therapeutically effective quantity of a compound of Formula I or one of its salts mixed with a pharmaceutically acceptable vehicle. The pharmaceutical formulations of the present invention can be liquids that are suitable for oral and/or parenteral administration, for example, drops, syrups, solutions, injectable solutions that are ready for use or are prepared by the dilution of a freeze-dried product but are preferably solid or semisolid as tablets, capsules, granules, powders, pellets, pessaries, suppositories, creams, salves, gels, ointments; or solutions, suspensions, emulsions, or other forms suitable for administration by the transdermal route or by inhalation.

The compounds of the invention can be administered for immediate-, delayed-, modified-, sustained-, pulsed-or controlled-release applications.

The most preferred oral compositions are slow, delayed or positioned release (e.g., enteric especially colonic release) tablets or capsules. This release profile can be achieved without limitation by use of a coating resistant to conditions within the stomach but releasing the contents in the colon or other portion of the GI tract wherein a lesion or inflammation site has been identified. Or a delayed release can be achieved by a coating that is simply slow to disintegrate. Or the two (delayed and positioned release) profiles can be combined in a single formulation by choice of one or more appropriate coatings and other excipients. Such formulations constitute a further feature of the present invention.

Suitable compositions for delayed or positioned release and/or enteric coated oral formulations include tablet formulations film coated with materials that are water resistant, pH sensitive, digested or emulsified by intestinal juices or sloughed off at a slow but regular rate when moistened. Suitable coating materials include, but are not limited to, hydroxypropyl methylcellulose, ethyl cellulose, cellulose acetate phthalate, polyvinyl acetate phthalate, hydroxypropyl methylcellulose phthalate, polymers of metacrylic acid and its esters, and combinations thereof. Plasticizers such as, but not limited to polyethylene glycol, dibutylphthalate, triacetin and castor oil may be used. A pigment may also be used to color the film. Suppositories are be prepared by using carriers like cocoa butter, suppository bases such as Suppocire C, and Suppocire NA50 (supplied by Gattefosse Deutschland GmbH, D-Weil am Rhein, Germany) and other Suppocire type excipients obtained by interesterification of hydrogenated palm oil and palm kernel oil (C8-C18 triglycerides), esterification of glycerol and specific fatty acids, or polyglycosylated glycerides, and whitepsol (hydrogenated plant oils derivatives with additives). Enemas are formulated by using the appropriate active compound according to the present invention and solvents or excipients for suspensions. Suspensions are produced by using micronized compounds, and appropriate vehicle containing suspension stabilizing agents, thickeners and emulsifiers like carboxymethylcellulose and salts thereof, polyacrylic acid and salts thereof, carboxyvinyl polymers and salts thereof, alginic acid and salts thereof, propylene glycol alginate, chitosan, hydroxypropylcellulose, hydroxypropyl-methylcellulose, hydroxyethylcellulose, ethylcellulose, methylcellulose, polyvinyl alcohol, polyvinyl pyrolidone, N-vinylacetamide polymer, polyvinyl methacrylate, polyethylene glycol, pluronic, gelatin, methyl vinyl ether-maleic anhydride copolymer, soluble starch, pullulan and a copolymer of methyl acrylate and 2-ethylhexyl acrylate lecithin, lecithin derivatives, propylene glycol fatty acid esters, glycerin fatty acid esters, sorbitan fatty acid esters, polyoxyethylene sorbitan fatty acid esters, polyethylene glycol fatty acid esters, polyoxyethylene hydrated caster oil, polyoxyethylene alkyl ethers, and pluronic and appropriate buffer system in pH range of 6.5 to 8. The use of preservatives, masking agents is suitable. The average diameter of micronized particles can be between 1 and 20 micrometers, or can be less than 1 micrometer. Compounds can also be incorporated in the formulation by using their water-soluble salt forms.

Alternatively, materials may be incorporated into the matrix of the tablet e.g. hydroxypropyl methylcellulose, ethyl cellulose or polymers of acrylic and metacrylic acid esters. These latter materials may also be applied to tablets by compression coating.

Pharmaceutical compositions can be prepared by mixing a therapeutically effective amount of the active substance with a pharmaceutically acceptable carrier that can have different forms, depending on the way of administration. Pharmaceutical compositions can be prepared by using conventional pharmaceutical excipients and methods of preparation. The forms for oral administration can be capsules, powders or tablets where usual solid vehicles including lactose, starch, glucose, methylcellulose, magnesium stearate, di-calcium phosphate, mannitol may be added, as well as usual liquid oral excipients including, but not limited to, ethanol, glycerol, and water. All excipients may be mixed with disintegrating agents, solvents, granulating agents, moisturizers and binders. When a solid carrier is used for preparation of oral compositions (e.g., starch, sugar, kaolin, binders disintegrating agents) preparation can be in the form of powder, capsules containing granules or coated particles, tablets, hard gelatin capsules, or granules without limitation, and the amount of the solid carrier can vary (between 1 mg to 1 g). Tablets and capsules are the preferred oral composition forms.

Pharmaceutical compositions containing compounds of the present invention may be in any form suitable for the intended method of administration, including, for example, a solution, a suspension, or an emulsion. Liquid carriers are typically used in preparing solutions, suspensions, and emulsions. Liquid carriers contemplated for use in the practice of the present invention include, for example, water, saline, pharmaceutically acceptable organic solvent(s), pharmaceutically acceptable oils or fats, and the like, as well as mixtures of two or more thereof. The liquid carrier may contain other suitable pharmaceutically acceptable additives such as solubilizers, emulsifiers, nutrients, buffers, preservatives, suspending agents, thickening agents, viscosity regulators, stabilizers, and the like. Suitable organic solvents include, for example, monohydric alcohols, such as ethanol, and polyhydric alcohols, such as glycols. Suitable oils include, for example, soybean oil, coconut oil, olive oil, safflower oil, cottonseed oil, and the like. For parenteral administration, the carrier can also be an oily ester such as ethyl oleate, isopropyl myristate, and the like. Compositions of the present invention may also be in the form of microparticles, microcapsules, liposomal encapsulates, and the like, as well as combinations of any two or more thereof.

Examples of pharmaceutically acceptable disintegrants for oral compositions useful in the present invention include, but are not limited to, starch, pre-gelatinized starch, sodium starch glycolate, sodium carboxymethylcellulose, croscarmellose sodium, microcrystalline cellulose, alginates, resins, surfactants, effervescent compositions, aqueous aluminum silicates and crosslinked polyvinylpyrrolidone.

Examples of pharmaceutically acceptable binders for oral compositions useful herein include, but are not limited to, acacia; cellulose derivatives, such as methylcellulose, carboxymethylcellulose, hydroxypropylmethylcellulose, hydroxypropylcellulose or hydroxyethylcellulose; gelatin, glucose, dextrose, xylitol, polymethacrylates, polyvinylpyrrolidone, sorbitol, starch, pre-gelatinized starch, tragacanth, xanthane resin, alginates, magnesium-aluminum silicate, polyethylene glycol or bentonite.

Examples of pharmaceutically acceptable fillers for oral compositions include, but are not limited to, lactose, anhydrolactose, lactose monohydrate, sucrose, dextrose, mannitol, sorbitol, starch, cellulose (particularly microcrystalline cellulose), dihydro- or anhydro-calcium phosphate, calcium carbonate and calcium sulfate.

Examples of pharmaceutically acceptable lubricants useful in the compositions of the invention include, but are not limited to, magnesium stearate, talc, polyethylene glycol, polymers of ethylene oxide, sodium lauryl sulfate, magnesium lauryl sulfate, sodium oleate, sodium stearyl fumarate, and colloidal silicon dioxide.

Examples of suitable pharmaceutically acceptable odorants for the oral compositions include, but are not limited to, synthetic aromas and natural aromatic oils such as extracts of oils, flowers, fruits (e.g., banana, apple, sour cherry, peach) and combinations thereof, and similar aromas. Their use depends on many factors, the most important being the organoleptic acceptability for the population that will be taking the pharmaceutical compositions.

Examples of suitable pharmaceutically acceptable dyes for the oral compositions include, but are not limited to, synthetic and natural dyes such as titanium dioxide, beta-carotene and extracts of grapefruit peel.

Suitable examples of pharmaceutically acceptable sweeteners for the oral compositions include, but are not limited to, aspartame, saccharin, saccharin sodium, sodium cyclamate, xylitol, malmitol, sorbitol, lactose and sucrose.

Suitable examples of pharmaceutically acceptable buffers include, but are not limited to, citric acid, sodium citrate, sodium bicarbonate, dibasic sodium phosphate, magnesium oxide, calcium carbonate and magnesium hydroxide.

Suitable examples of pharmaceutically acceptable surfactants include, but are not limited to, sodium lauryl sulfate and polysorbates.

Suitable examples of pharmaceutically acceptable preservatives include, but are not limited to, various antibacterial and antifungal agents such as solvents, for example ethanol, propylene glycol, benzyl alcohol, chlorobutanol, quaternary ammonium salts, and parabens (such as methyl paraben, ethyl paraben, propyl paraben, etc.).

Suitable examples of pharmaceutically acceptable stabilizers and antioxidants include, but are not limited to, ethylenediaminetetraacetic acid (EDTA), thiourea, tocopherol and butyl hydroxyanisole.

The compounds of the invention may also, for example, be formulated as suppositories e.g., containing conventional suppository bases for use in human or veterinary medicine or as pessaries e.g., containing conventional pessary bases.

The compounds according to the invention may be formulated for topical administration, for use in human and veterinary medicine, in the form of ointments, creams, gels, hydrogels, lotions, solutions, shampoos, powders (including spray or dusting powders), pessaries, tampons, sprays, dips, aerosols, drops (e.g., eye ear or nose drops) or pour-ons.

For application topically to the skin, the agent of the present invention can be formulated as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax, sorbitan monostearate, a polyethylene glycol, liquid paraffin, polysorbate 60, cetyl esters wax, cetearyl alcohol, 2-octyldodecanol, benzyl alcohol, and water. Such compositions may also contain other pharmaceutically acceptable excipients, such as polymers, oils, liquid carriers, surfactants, buffers, preservatives, stabilizers, antioxidants, moisturizers, emollients, colorants, and odorants.

Examples of pharmaceutically acceptable polymers suitable for such topical compositions include, but are not limited to, acrylic polymers; cellulose derivatives, such as carboxymethylcellulose sodium, methylcellulose or hydroxypropylcellulose; natural polymers, such as alginates, tragacanth, pectin, xanthan and cytosan.

As indicated, the compound of the present invention can be administered intranasally or by inhalation and is conveniently delivered in the form of a dry powder inhaler or an aerosol spray presentation from a pressurized container, pump, spray or nebulizer with the use of a suitable propellant, e.g., dichlorodifluoromethane, trichlorofluoromethane, dichlorotetrafluoroethane, a hydrofluoroalkane such as 1,1,1,2-tetrafluoroethane (HFA 134AT″″) or 1,1,1,2,3,3,3-heptafluoropropane (HFA 227EA), carbon dioxide or other suitable gas. In the case of a pressurized aerosol, the dosage unit may be determined by providing a valve to deliver a metered amount. The pressurized container, pump, spray or nebulizer may contain a solution or suspension of the active compound, e.g., using a mixture of ethanol and the propellant as the solvent, which may additionally contain a lubricant, e.g., sorbitan trioleate.

Capsules and cartridges (made, for example, from gelatin) for use in an inhaler or insufflator may be formulated to contain a powder mix of the compound and a suitable powder base such as lactose or starch. For topical administration by inhalation the compounds according to the invention may be delivered for use in human or veterinary medicine via a nebulizer.

The pharmaceutical compositions of the invention may contain from 0.01 to 99% weight per volume of the active material. For topical administration, for example, the composition will generally contain from 0.01-10%, more preferably 0.01-1% of the active material.

The therapeutically effective quantities will depend on the age and on the general physiological condition of the patient, the route of administration and the pharmaceutical formulation used. The therapeutic doses will generally be between about 10 and 2000 mg/day and preferably between about 30 and 1500 mg/day. Other ranges may be used, including, for example, 50-500 mg/day, 50-300 mg/day, 100-200 mg/day.

Administration may be once a day, twice a day, or more often, but frequency may be decreased during a maintenance phase of the disease or disorder, e.g. once every second or third day instead of every day or twice a day. The dose and the administration frequency will depend on the clinical signs, which confirm maintenance of the remission phase, with the reduction or absence of at least one or more preferably more than one clinical signs of the acute phase known to the person skilled in the art. More generally, dose and frequency will depend in part on recession of pathological signs and clinical and subclinical symptoms of a disease condition or disorder contemplated for treatment with the present compounds.

The duration of the treatment can range from weeks to months to years as long as benefits persist and/or side-effects are tolerated, and/or inflammation or its markers is abated (lessened or eliminated).

### EXAMPLES

The therapeutic effect of compounds of the present invention was determined in in vitro and in vivo experiments such as the following.

The cytokines assayed in the biological examples, when expressed at elevated amounts, are markers for inflammation and, in the case of cell proliferation, granulocyte degranulation and lung neutrophilia, the behaviors of these immune cells are also markers for their activation and, therefore, inflammation. Consequently, reduction of pro-inflammatory cytokine expression or secretion and reduction in cell proliferation, degranulation or neutrophil accumulation is a measure of a compound's anti-inflammatory activity. Lung neutrophilia specifically serves as a model for COPD.

A compound analyzed using the biological assays as defined herein is considered to be “active” if it is better than a positive control (e.g., azithromycin) in at least one inhibitory function (i.e., inhibition of TNF-α or IL-6) after stimulation with at least one stimulant (e.g., PMA or PHA). Preferably a compound should exhibit more than 50% inhibition in at least one assay at a concentration that is non-toxic.

### Example 1

### Sample Preparation

Test substances used in in vitro experiments were dissolved in dimethyl sulfoxide (DMSO) (Kemika, Croatia) at concentrations of 50 mM and 10 mM, and were further diluted to final concentrations of 50 μM and 10 μM in 1 mL Dulbecco's modified Eagle medium (DMEM) supplemented with 1% heat inactivated fetal bovine serum (FBS), 1% L-glutamine, 50 U/ml penicillin, 50 μg/ml streptomycin and 2.5 μg/mL Fungizone (amphotericin B). Media and all media supplements were purchased from Gibco, Australia, except the FBS was purchased from Sigma, USA.

### Example 2

### Isolation of Peripheral Blood Leukocytes

Peripheral blood leukocytes (PBL) were obtained from venous blood of healthy volunteers by sedimentation on 2% dextran T-500 (Amersham Biosciences, USA) and subsequent centrifugations of leukocyte rich plasma.

### Example 3

### Inhibition of Proinflammatory Cytokine Production by Stimulated Human Peripheral Blood Leukocytes In Vitro

Peripheral blood leukocytes (PBL), isolated as described above, were seeded in a 48-well plate at a concentration of 3 to 5×10⁶ cell per well in culture medium consisting of RPMI 1640 medium (Institute of Immunology, Croatia) supplemented with 10% heat-inactivated fetal calf serum (FCS, Biowhittaker, USA), 100 U/ml penicillin (Gibco, Australia), 100 μg/ml streptomycin (Gibco, Australia) and 2 mM L-glutamine (Gibco, Australia), and preincubated with the test compounds for 2 h at 37° C., in an atmosphere of 5% CO₂, and 90% humidity. Then, stimuli (Sigma, USA) were added to a final concentration of 2 μg/mL lipopolysaccharide (LPS), 1 μg/mL phorbol 12-myristate 13-acetate (PMA) or 120 μg/mL zymosan. Samples were incubated overnight under conditions described above. At the end of incubation, the supernatants were transferred to eppendorf tubes and centrifuged for 10 min at 1500×g. Concentrations of human TNF-α, IL-1β, IL-6 and IL-8 were determined in cell supernatants by sandwich ELISA, using capture and detection antibodies (R&D Systems, Minneapolis, Minn.) according to the manufacturer's recommendations.

Inhibition (as percentage) was calculated using the following formula: % inhibition=(1−concentration of cytokines in sample/concentration of cytokines in positive control)×100. The positive control refers to stimulated samples, not treated with substances.

TABLE 1
Percentage of inhibition of proinflammatory cytokine production by stimulated PBL treated with compounds

| | | TNF-α | | | IL-1α | | | IL-6 | | | IL-8 | | |
|---|---|---|---|---|---|---|---|---|---|---|---|---|---|
| | | LPS | PMA | zymosan | LPS | PMA | zymosan | LPS | PMA | zymosan | LPS | PMA | zymosan |
| Azithromycin | 10 μM | 36 | 0 | 6 | 28 | 0 | 20 | 15 | 0 | 20 | 0 | 0 | 0 |
| | 50 μM | 20 | 0 | 64 | 48 | 0 | 75 | 40 | 0 | 75 | 0 | 0 | 0 |
| Clarithromycin | 10 μM | 0 | 0 | 0 | 0 | 26 | 36 | 11 | 0 | 28 | 23 | 33 | 0 |
| | 50 μM | 0 | 0 | 0 | 0 | 33 | 59 | 0 | 0 | 0 | 0 | 0 | 0 |
| Compound 1 | 10 μM | 0 | 2 | 34 | 52 | 38 | 65 | 4 | 22 | 23 | 40 | 4 | 12 |
| | 50 μM | 33 | 37 | 72 | 78 | 47 | 57 | 22 | 21 | 59 | 0 | 0 | 79 |
| Compound 2 | 10 μM | 37 | 60 | 78 | 77 | 18 | 92 | 75 | 49 | 90 | 79 | 27 | 61 |
| | 50 μM | 100 | 99 | 98 | 98 | 97 | 98 | 100 | 100 | 100 | 88 | 58 | 51 |
| Compound 3 | 10 μM | 45 | 37 | 71 | 52 | 4 | 75 | 13 | 24 | 59 | 38 | 61 | 40 |
| | 50 μM | 74 | 75 | 94 | 30 | 0 | 74 | 79 | 9 | 81 | 81 | 47 | 88 |
| Compound 4 | 10 μM | 39 | 50 | 36 | 74 | 54 | 60 | 38 | n.d. | 0 | 41 | 52 | 0 |
| | 50 μM | 100 | 100 | 97 | 82 | 92 | 13 | 100 | n.d. | 100 | 100 | 100 | 52 |
| Compound 5 | 10 μM | 19 | 62 | 49 | 41 | 14 | 27 | 64 | 44 | 66 | 16 | 23 | 20 |
| | 50 μM | 45 | 85 | 76 | 10 | 14 | 41 | 72 | 60 | 88 | 42 | 69 | 46 |
| Compound 6 | 10 μM | 74 | 73 | 36 | 64 | 79 | 67 | 59 | n.d. | 6 | 43 | 23 | 41 |
| | 50 μM | 100 | 100 | 98 | 98 | 100 | 89 | 100 | n.d. | 100 | 100 | 100 | 71 |
| Compound 7 | 10 μM | 0 | 95 | 69 | 71 | 0 | 96 | 54 | 65 | 73 | 39 | 40 | 61 |
| | 50 μM | 100 | 100 | 100 | 89 | 79 | 83 | 100 | 90 | 100 | 100 | 100 | 99 |
| Compound 8 | 10 μM | 58 | 56 | 16 | 42 | 26 | 16 | 60 | n.d. | 20 | 48 | 37 | 0 |
| | 50 μM | 30 | 54 | 0 | 40 | 0 | 17 | 82 | n.d. | 18 | 0 | 0 | 0 |
| Compound 9 | 10 μM | 23 | n.d. | 39 | 45 | n.d. | 65 | 0 | n.d. | 58 | 0 | n.d. | 0 |
| | 50 μM | 100 | n.d. | 100 | 43 | n.d. | 22 | 100 | n.d. | 100 | 100 | n.d. | 100 |
| Compound 10 | 10 μM | 32 | 0 | 69 | 0 | 0 | 0 | 63 | 40 | 76 | 55 | 75 | 82 |
| | 50 μM | 99 | 100 | 99 | 100 | 100 | 96 | 100 | 100 | 100 | 93 | 100 | 97 |
| Compound 11 | 10 μM | 0 | n.d. | 13 | 48 | n.d. | 85 | 0 | n.d. | 72 | 0 | n.d. | 23 |
| | 50 μM | 51 | n.d. | 88 | 38 | n.d. | 77 | 66 | n.d. | 100 | 52 | n.d. | 75 |
| Compound 12 | 10 μM | 28 | n.d. | 70 | 52 | n.d. | 80 | 6 | n.d. | 88 | 15 | n.d. | 40 |
| | 50 μM | 100 | n.d. | 100 | 82 | n.d. | 100 | 100 | n.d. | 100 | 100 | n.d. | 100 |
| Compound 13 | 10 μM | 0 | 0 | 0 | 0 | 0 | 34 | 0 | 84 | 52 | 0 | 0 | 62 |
| | 50 μM | 0 | 0 | 52 | 52 | 32 | 89 | 0 | 100 | 94 | 0 | 25 | 0 |

### Example 4

### Isolation of Peripheral Blood Mononuclear Cells

Heparinized peripheral blood was obtained from healthy donors, and peripheral blood mononuclear cells (PBMC) were isolated by Histopaque 1077 (Sigma, USA) density centrifugation on 400 g for 30 minutes. Collected PBMC were centrifuged in plasma on 400 g/10 min, resuspended and washed in RPMI 1640 (Institute of Immunology, Croatia) by centrifugation.

### Example 5

### Inhibition of Production of T-Cell Specific Cytokines, IL-2 and IL-5, by Stimulated Human Peripheral Blood Mononuclear Cells In Vitro

Peripheral blood mononuclear cells (PBMC), isolated as described above, were seeded in a 48-well plate at a concentration of 1×10⁶ cells per well in the RPMI culture medium described above. Cells were stimulated with 10 μg/mL phytohaemagglutinin (PHA) (Sigma, USA) and incubated at 37° C., 5% CO₂ in 90% humidity with tested compounds (10 and 50 μM) for 3 days. Cytokine concentrations were determined in supernatants by sandwich ELISA, using capture and detection antibodies (R&D, USA) according to the manufacturer's recommendations.

Inhibition (as percentage) was calculated using the following formula: % inhibition=(1−concentration of cytokines in sample/concentration of cytokines in positive control)×100. The positive control refers to stimulated samples, not treated with substances.

TABLE 2
Percentage of inhibition of IL-2 and IL-5 production by stimulated
PBMC treated with compounds

| | | IL-2 | IL-5 |
|---|---|---|---|
| clarithromycin | 10 μM | 0 | 13 |
| | 50 μM | 3 | 47 |
| Compound 1 | 10 μM | 61 | 14 |
| | 50 μM | 95 | 73 |
| Compound 3 | 10 μM | 86 | 80 |
| | 50 μM | 98 | 78 |

### Example 6

### Effects on Human T-Cell Proliferation In Vitro

Influence of substances at two different concentrations (50 μM and 10 μM) on cell proliferation of human peripheral blood mononuclear cells (PBMC) was assessed.

Heparinized peripheral blood was obtained from healthy donors, and PBMC were isolated by Histopaque 1077 (Sigma, USA) density centrifugation at 400×g for 30 minutes. 5×10⁴ cells/well were cultured for 3 days in the RPMI medium as described above, in the presence (positive control) or absence (negative control) of stimulators [PHA (2.5 μg/mL) (Sigma, USA), or both PMA (10 ng/mL) (Sigma, USA) and ionomycin (500 ng/mL) (Calbiochem, USA)], and in the presence of the test compounds, at 37° C. in an atmosphere of 5% CO₂ and 90% humidity. The cells were pulsed with 1 μCi of ³H-thymidine (Amersham, USA) per well during the last 18 h of the culture, and were harvested on the 96-well filter (Packard Bioscience, USA) using a multiple cell harvester (Packard, USA). The incorporation of ³H-thymidine in activated cells were measured using TopCount NXT (Packard, USA).

Inhibition (as percentage) was calculated using the following formula: % inhibition=(1−(³H)thymidine incorporation expressed in counts per minute (cpm) in sample/(³H)thymidine incorporation expressed in cpm in positive control)×100

where positive control refers to stimulated samples, not treated with substances.

TABLE 3
Percentage of inhibition of proliferation of stimulated PBMC cell
line treated with compounds

PBMC

| | | PHA | PMA + ionomycin |
|---|---|---|---|
| Azithromycin | 10 μM | 5 | 9 |
| | 50 μM | 29 | 31 |
| Clarithromycin | 10 μM | 0 | 9 |
| | 50 μM | 7 | 14 |
| Compound 1 | 10 μM | 21 | 10 |
| | 50 μM | 95 | 52 |
| Compound 2 | 10 μM | 6 | 35 |
| | 50 μM | 97 | 99 |
| Compound 3 | 10 μM | 26 | 23 |
| | 50 μM | 100 | 92 |
| Compound 4 | 10 μM | 30 | 50 |
| | 50 μM | 102 | 100 |
| Compound 5 | 10 μM | 0 | 20 |
| | 50 μM | 72 | 87 |
| Compound 6 | 10 μM | 0 | 0 |
| | 50 μM | 100 | 100 |
| Compound 8 | 10 μM | 0 | 0 |
| | 50 μM | 0 | 0 |
| Compound 13 | 10 μM | 11 | 21 |
| | 50 μM | 42 | 43 |

### Example 7

### Isolation of Granulocytes

Granulocytes were obtained from heparinized whole blood using density gradient centrifugation. Erythrocytes were sedimented on 3% dextran T-500 (Amersham Pharmacia Biotech AB, Uppsala, Sweden). Leukocytes were centrifuged on Ficoll (Amersham Pharmacia Biotech AB, Uppsala, Sweden) for 35 min at 600 g at 20° C. The pellet of granulocytes was cleared from remaining erythrocytes by a brief hypotonic lysis.

### Example 8

### Inhibition of Degranulation of Granulocytes

1×10⁶ granulocytes, isolated as described above, were resuspended in RPMI-1640 medium (Institute of Immunology, Croatia), and were incubated with 10 or 50 μM of test compound together with cytochalasine B (5 μg/mL) for 2 h at 37° C. Degranulation then was induced by the addition of 0.1 μM fMLP (Sigma, USA) or 0.5 μM A23187 (Calbiochem, USA). The activity of free neutrophil elastase was determined with a chromogenic substrate specific for human neutrophil elastase, such as n-methoxysuccinyl-1-alanyl-1-alanyl-1-prolyl-lvalyl-p-niytoanlide (Sigma Chemical Company, St Louis, Mo., USA) Elastase activity, as a marker of primary granules, was assessed in supernatant using a spectrophotometer at an absorbance of 405 nm. Results are expressed as the percent of inhibition of degranulation in unstimulated cells, and in stimulated cells treated with fMLP or A23187.

TABLE 4
Percentage of inhibition of degranulation of granulocytes
stimulated with fMLP or A23187

| | | fMLP | A23187 |
|---|---|---|---|
| | | 0.1 μM | 0.5 μM |
| azithromycin | 50 μM | 36 | 16 |
| erythromycin | 50 μM | 23 | 0 |
| Compound 1 | 10 μM | 37 | 20 |
| | 50 μM | 72 | 27 |
| Compound 3 | 10 μM | 25 | 2 |
| | 50 μM | 33 | 75 |

### Example 9

### Accumulation in Granulocytes

7.5×10⁶ granulocytes, isolated as described above, were suspended in 3 mL of RPMI 1640 (Institute of Immunology, Croatia) containing 10 μM of tested macrolide. Samples were incubated at 37° C. for 180 min. Following incubation, the samples were centrifuged through the layer of Poly(dimethylsiloxane-co-diphenylsiloxane), dihydroxy terminated—silicone oil (Aldrich Chemical Company, Milwaukee, USA). The pellet was resuspended in 0.5% Triton X-100 (Sigma, St. Louis, USA) in deionized water (MilliQ, Millipore Corporation, Bedford, USA). The suspension was sonicated, proteins were precipitated with acetonitrile, and the concentration of macrolide in the supernatants was determined by liquid chromatography-mass spectrometry (LC-MS).

Intracellular concentrations of the test compounds were calculated from the mean cell number recovered after centrifuging through silicone oil. According to the literature, about 1 million neutrophils is considered to have a volume of 0.24 μL (Vazifeh et al., Antimicrob Agents Chemo. 1997; 41: 2099-2107). To estimate the extent of macrolide accumulation, the intracellular to extracellular concentration ratio (I/E) was calculated, where E (due to a large volume of incubation medium) was taken as constant (10 μM). Results are expressed relative to azithromycin according to the following equation: % of azithromycin uptake=(I/E of a substance/I/E of azithromycin)×100. I/E values obtained for azithromycin were 164±10.

TABLE 5
Accumulation of substances in granulocytes
expressed relative to azithromycin uptake

| | % of azithromycin uptake |
|---|---|
| azithromycin | 100 |
| Compound 1 | 93 |
| Compound 3 | 93 |

### Example 10

### Cytotoxicity Assay on Hep G2 and A549 Cell Lines

To determine whether the anti-inflammatory activity of the test compounds was due to observed inhibition of cytokine production and inhibition of proliferation in vitro, and was not a consequence of cellular cytotoxicity, measurement of succinate dehydrogenase activity in living cells was performed. Cells were cultured for 24 h in RPMI medium as described above at 37° C. in the presence of the test compounds at concentrations of 50 μM and 12.5 μM. MTT [3-(4,5-dimethylthiazole-2-yl)-2,5-diphenyl tetrazolium bromide] (Promega, USA), a detection reagent, was then added and the cultures were incubated for 0.5-2 h. The amount of MTT-Formazan produced was determined using a spectrophotometer at 490 nm (Mosmann, J. Imnunol. Methods, 1983, 65: 55-63). Percentage of viable cells was calculated using the following formula:

% viable cells=(1−OD₄₉₀ treated cells/OD₄₉₀ nontreated cells)×100

TABLE 6
Percentage of viable cells after treatment with substances

| | | | A549 | Hep G2 |
|---|---|---|---|---|
| Azithromycin | 12.5 | μM | 100 | 100 |
| | 50 | μM | 100 | >95 |
| Clarithromycin | 12.5 | μM | 100 | 100 |
| | 50 | μM | 100 | 100 |
| Compound 1 | 12.5 | μM | n.d. | 100 |
| | 50 | μM | n.d. | >95 |
| Compound 2 | 12.5 | μM | 100 | >85 |
| | 50 | μM | >70 | 5 |
| Compound 3 | 12.5 | μM | 100 | 100 |
| | 50 | μM | >90 | >85 |
| Compound 5 | 12.5 | μM | 100 | 100 |
| | 50 | μM | 100 | 100 |
| Compound 7 | 12.5 | μM | 100 | 100 |
| | 50 | μM | 88 | 20 |
| Compound 9 | 12.5 | μM | 100 | 92 |
| | 50 | μM | 100 | 21 |
| Compound 10 | 12.5 | μM | 100 | 100 |
| | 50 | μM | 70 | 8 |
| Compound 11 | 12.5 | μM | 100 | 100 |
| | 50 | μM | 85 | 95 |
| Compound 12 | 12.5 | μM | 94 | 93 |
| | 50 | μM | 72 | 8 |

### Example 11

### Lipopolysaccharide Induced TNF-α Production in Balb/cJ Mice

Male Balb/cJ mice (Iffa Credo, France), 18 animals weighing 25-33 g were randomly grouped (7 animals were treated with the test compounds, 7 were in positive control and 4 in negative control). Substances, as well as vehiculum [0.125% carboxymethyl-cellulose (Sigma, USA)], were administered intraperitoneally. Substances were administered in the dose of 10 mg/kg b.w. Administered volume was 10 mL/kg b.w. Thirty minutes later, lipopolysaccharide (LPS) (Sigma, USA) solution in sterile saline at concentration and volume of 25 μg/0.2 mL of sterile saline/mouse was intraperitoneally administered to each animal except those in negative control. Ninety minutes after the challenge all animals were bled to death by puncturing a carotis communis. Plasma concentration of TNF-α was determined by sandwich ELISA, using capture and detection antibodies (R&D, USA) according to manufacturer's recommendations. Results are presented in the following table as % of inhibition of TNF-α production in comparison to positive control (challenged but not treated animals).

TABLE 7
Percentage of inhibition of TNF-α production in LPS stimulated
Balb/cJ mice treated with compounds

| | TNF-α |
|---|---|
| Compound | (% inhibition) |
| azithromycin | 54 |
| clarithromycin | 63 |
| Compound 1 | 31 |
| Compound 3 | 29 |
| Compound 5 | 50 |

### Example 12

### Oxidative Burst Inhibition Assay on Granulocytes

The effect of compounds on oxidative burst was investigated in vitro with oxidative burst assay in human granulocytes, isolated as described above. Assay was done in 96 well plate. Blood was obtained from healthy volunteers (Transfusion Center, Croatia) and human granulocytes were isolated by dextran sedimentation and ficoll extraction method, as described above. In each well 50000 isolated granulocytes were suspended in 100 μL of DMEM. Final concentrations of the tested compounds were 50 and 12.5 μM. Than, 50 μL of luminol (c=0.25 mg/mL) was added. Luminol as a detector of oxidative burst react with superoxide and light is produced as one of the reaction products. Subsequently, 50 μL of stimuli, phorbol-12-myristate acetate (PMA) (c=33 ng/mL) or Zymosan (c=120 μg/mL), were added.

TABLE 8
Percentage of oxidative burst inhibition on Zymosan or PMA
stimulated granulocytes treated with compounds

| | | | % inhibition of | |
|---|---|---|---|---|
| | | | Oxidative burst | % inhibition of Oxidative |
| | | | Zymosan | burst PMA |
| Azithromycin | 12.5 | μM | 0 | 0 |
| | 50 | μM | 16 | 20 |
| Erythromycin | 12.5 | μM | 6 | 0 |
| | 50 | μM | 41 | 0 |
| Clarithromycin | 12.5 | μM | 4 | 0 |
| | 50 | μM | 19 | 3 |
| Compound 2 | 12.5 | μM | 24 | 19 |
| | 50 | μM | 98 | 82 |
| Compound 4 | 12.5 | μM | 0 | 16 |
| | 50 | μM | 100 | 85 |

### Example 13

### Lung Neutrophilia Induced by Bacterial Lipopolysaccharide in Male BALB/cJ Mice

Male BALB/cJ mice (Iffa Credo, France), with average weight ˜30 g were randomly grouped (n=7 in testing group, 8 in positive control, 7 in negative control). Mice were administered intraperitoneally (i.p.) a single 5 mg dose of test compound. Two hours after administration, 2 μg of bacterial lipopolysaccharide (LPS), dissolved in PBS in a volume of 60 μL, was intranasally administered to all experimental groups except the negative control group, which received the same volume (60 μL) of vehicle PBS. Animals were sacrificed approximately 24 hours after application of LPS in order to obtain bronchoalveolar lavage fluid (BALF), which was used to determine concentrations of IL-6 and TNF-α, absolute number of cells, and the percentage of neutophils in BALF. Results are expressed as percentage of decrease of total cell number, relative number of neutrophils, and TNF-α and IL-6 concentration in BALF of treated animals compared to positive control (LPS stimulated, but untreated animals).

| | | | |
|---|---|---|---|
| Total cell no. | % of neutrophils | TNF-α | IL-6 |

| | | | | |
|---|---|---|---|---|
| Azithromycin | 77 | 37 | 66 | 72 |
| Clarithromycin | 77 | 36 | 78 | 60 |
| Compound 1 | 50 | 9 | 41 | 28 |

Besides accumulation of inflammatory cells in BALF, the extent and anatomic site of pulmonary inflammation induced by LPS was assessed 24 hours after PBS or LPS exposure. Accumulation of granulocytes and mononuclear cells in peribronchial (PB) and perivascular (PV) lung tissue areas and in alveolar spaces was monitored following sacrifice of the animals.

Challenge with LPS induced significant accumulation of both granulocytes and mononuclear cells in lung tissue in comparison to groups challenged with PBS (negative control). The tested compounds significantly decreased accumulation of both granulocytes and mononuclears in lung tissue (PB and PV).

In all the tests, the compounds of the present invention were found to be very active as anti-inflammatories and the anti-inflammatory activity was found to be comparable or greater than that of the comparative compounds.

What is claimed is:

1. A method of treatment of an inflammatory disease, disorder or condition characterized by or associated with an undesirable inflammatory immune response, which comprises administering to a subject a therapeutically effective amount of a compound of Formula (I) 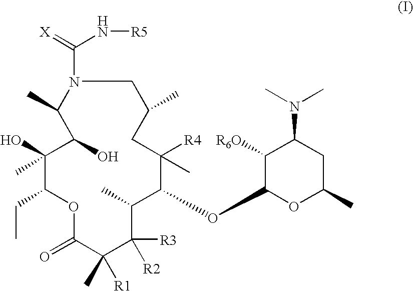
wherein
R¹ is hydrogen or together with R² is a double bond;
R² is a cladinose sugar of formula (II), hydrogen, hydroxyl or group of the formula (III) wherein Y is a monocyclic aromatic ring unsubstituted or substituted with groups which are selected from halogen, OH, OMe, NO₂, and NH₂; or 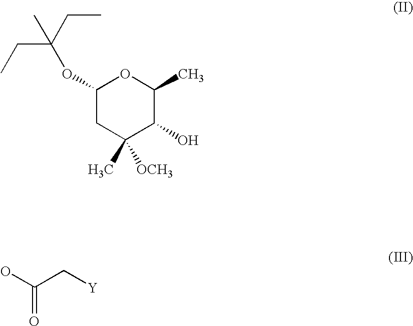
R² together with R³ is a ketone, or together with R¹ is a double bond;
R³ is hydrogen or together with R² is a ketone, or together with R⁴ is an ether;
R⁴ is hydroxyl or OMe, or together with R³ is an ether;
R⁵ is C1-4alkyl, C2-4alkenyl, —(CH₂)ₘ—Ar, wherein Ar is a monocyclic or bicyclic aromatic ring having up to 10 carbon atoms, containing 0-3 heteroatoms selected from N and O, unsubstituted or substituted by halogen, C₁₋₆haloalkyl, C₁₋₆haloalkoxy, C₁₋₆alkyl, C₁₋₆alkoxy, and m is 0-3,; or
R⁶ is hydrogen or a hydroxyl protective group
X is oxygen or sulfur; or a
pharmaceutically acceptable salt thereof.

2. The method of claim 1, wherein
R¹ is hydrogen;
R² is a cladinose sugar of formula (II) or hydroxyl 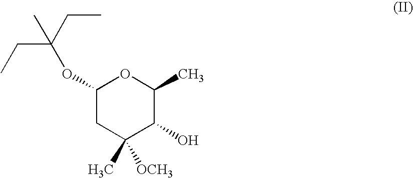
R³ is hydrogen or together with R⁴ represents an ether;
R⁴ is hydroxyl or together with R³ is an ether;
R⁵ is —(CH₂)ₘ—Ar, wherein Ar is a monocyclic or bicyclic aromatic ring up to 10 carbon atoms, unsubstituted or substituted by one or more of halogen, C₁₋₆haloalkyl, and m is 0-2,
R⁶ is hydrogen, and
X is oxygen or sulfur; or a
pharmaceutically acceptable salt thereof.

3. The method of claim 1, wherein
R¹ is hydrogen;
R² is a cladinose sugar of formula (II) or hydroxyl 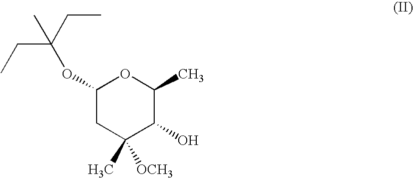
R³ is hydrogen or together with R⁴ represents an ether;
R⁴ is hydroxyl or together with R³ is an ether;
R⁵ is benzyl, 4-chlorophenyl, 3-fluorophenyl, 3-trifluoromethylphenyl, 2-fluorophenyl, 3-bromophenyl, 4-bromophenyl, 4-trifluoromethylphenyl, 3-trifluoromethyl-4-chlorophenyl, 1-(1-naphthyl)-ethyl,
R⁶ is hydrogen, and
X represents oxygen or sulfur; or a
pharmaceutically acceptable salt thereof.

4. A method of treating an inflammatory condition or an immune or anaphylactic disorder associated with infiltration of leukocytes into inflamed tissue in a subject in need thereof which comprises administering to said subject a therapeutically effective amount of the compound of Formula (I).

5. The method according to claim 1, wherein said condition, disorder, or disease is selected from the group consisting of asthma, COPD, diffuse panbronchiolitis, adult respiratory distress syndrome, inflammatory bowel disease, Crohn's disease, bronchitis, chronic sinusitis, pulmonary fibrosis, diffuse panbronchiolitis and cystic fibrosis.

6. A method according to claim 1, wherein said condition, disorder or disease is selected from the group consisting of inflammatory conditions and immune disorders of the lungs, joints, eyes, bowel, skin, and heart.

7. A method according to claim 1, wherein said condition, disorder, or disease is selected from the group consisting of asthma, adult respiratory distress syndrome, bronchitis, bronchiectasis, bronchiolitis obliterans, cystic fibrosis, rheumatoid arthritis, rheumatoid spondylitis, osteoarthritis, osteomyelitis, sinusitis, nasal polyps, gouty arthritis, uveitis, conjunctivitis, inflammatory bowel disease, Crohn's disease, ulcerative colitis, distal proctitis, psoriasis, eczema, dermatitis, acne, coronary infarct damage, coronary artery disease, chronic inflammation, endotoxin shock, and smooth muscle proliferation disorders.

8. A method of treatment of an inflammatory disease, disorder, or condition characterized by or associated with excessive unregulated production of inflammatory cytokines or inflammatory mediators which comprises administering to a subject a therapeutically effective amount of a compound according to Formula (I) effective to reduce or inhibit T-cell proliferation or cytokine production.

9. A method for treatment of a disease or disorder or condition associated with excessive secretion of one or more of TNF-α, IL-1, IL-6, IL-8, IL-2 or IL-5, which comprises a method of treatment of inflammatory diseases, disorders and conditions characterized by or associated with an undesirable inflammatory immune response, which comprises administering to a subject a therapeutically effective amount of a compound of Formula (I) 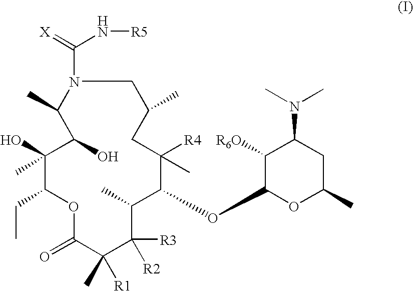
wherein
R¹ is hydrogen or together with R is a double bond;
R² is a cladinose sugar of formula (II), hydrogen, hydroxyl or group of the formula (III) wherein Y is a monocyclic aromatic ring unsubstituted or substituted with groups which are selected from halogen, OH, OMe, NO₂, and NH₂; or 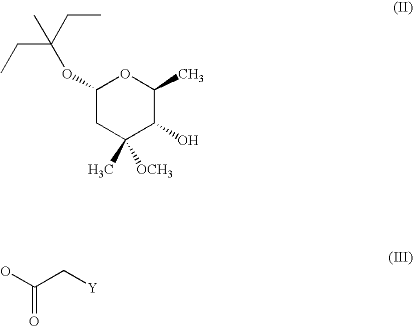
R² together with R³ is a ketone, or together with R¹ is a double bond;
R³ is hydrogen or together with R² is a ketone, or together with R⁴ is an ether;
R⁴ is hydroxyl or OMe, or together with R³ is an ether;
R⁵ is C1-4alkyl, C2-4alkenyl, —(CH₂)ₘ—Ar, wherein Ar is a monocyclic or bicyclic aromatic ring having up to 10 carbon atoms, containing 0-3 heteroatoms selected from N and O, unsubstituted or substituted by halogen, C₁₋₆haloalkyl, C₁₋₆haloalkoxy, C₁₋₆alkyl, C₁₋₆alkoxy, and m is 0-3,; or
R⁶ is hydrogen or a hydroxyl protective group
X is oxygen or sulfur; or a
pharmaceutically acceptable derivatives salt thereof.

10. The method of claim 9, wherein
R¹ is hydrogen;
R² is a cladinose sugar of formula (II) or hydroxyl 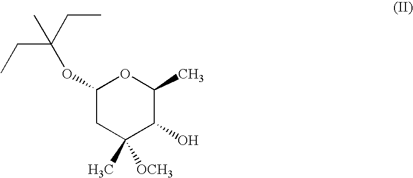
R³ is hydrogen or together with R⁴ represents an ether;
R⁴ is hydroxyl or together with R³ is an ether;
R⁵ is —(CH₂)ₘ—Ar, wherein Ar is a monocyclic or bicyclic aromatic ring up to 10 carbon atoms, unsubstituted or substituted by one or more of halogen, C₁₋₆haloalkyl, and m is 0-2,
R⁶ is hydrogen, and
X represents oxygen or sulfur; or a
pharmaceutically acceptable derivatives salt thereof.

11. The method of claim 9, wherein
R¹ is hydrogen;
R² is a cladinose sugar of formula (II) or hydroxyl 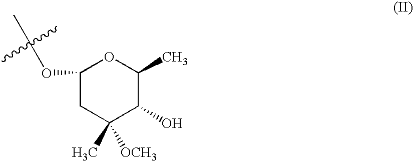
R³ is hydrogen or together with R⁴ represents an ether;
R⁴ is hydroxyl or together with R³ is an ether;
R⁵ is benzyl, 4-chlorophenyl, 3-fluorophenyl, 3-trifluoromethylphenyl, 2-fluorophenyl, 3-bromophenyl, 4-bromophenyl, 4-trifluoromethylphenyl, 3-trifluoromethyl-4-chlorophenyl, 1-(1-naphthyl)-ethyl,
R⁶ is hydrogen, and
X represents oxygen or sulfur; or a
pharmaceutically acceptable salt thereof.

12. The method of claim 1, wherein the compound of formula I is selected from the group consisting of: 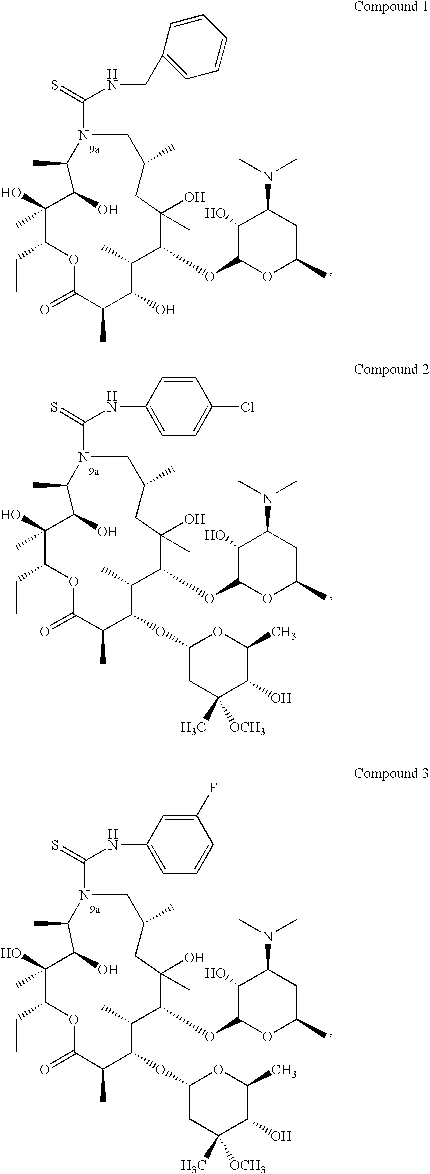 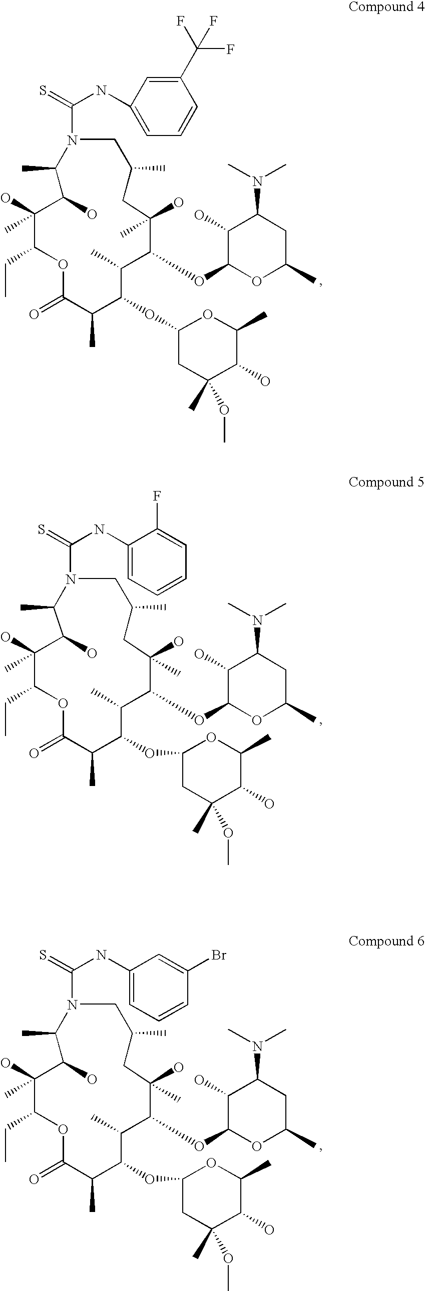 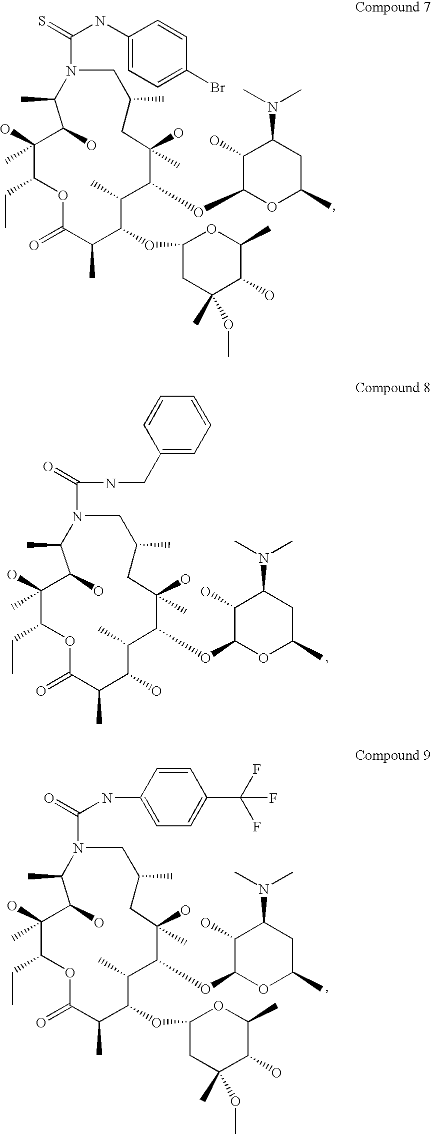 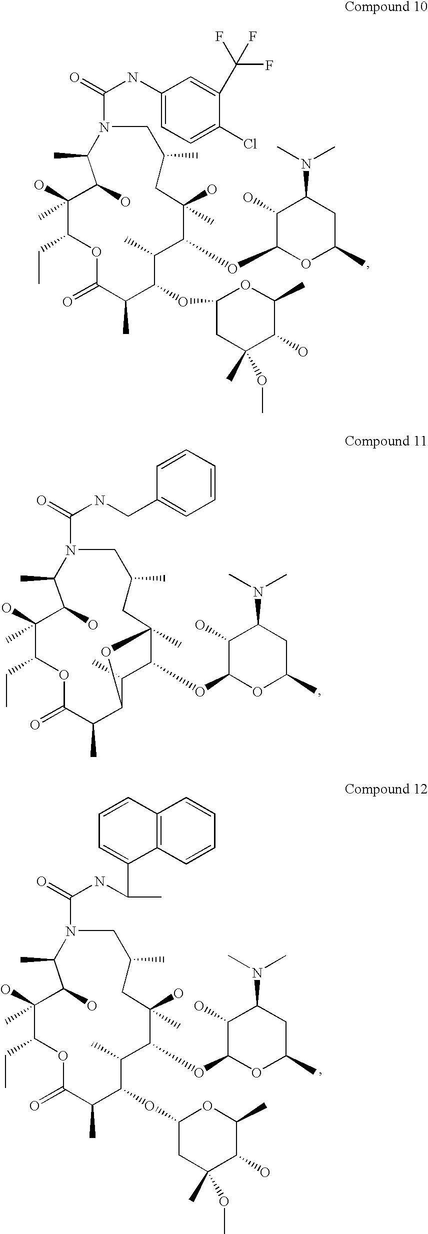 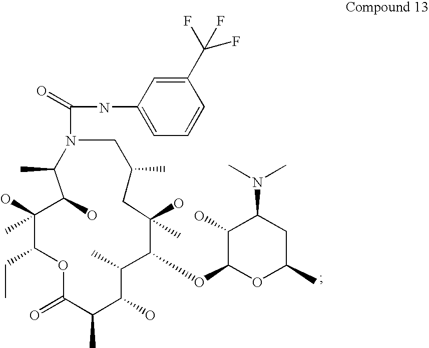
and pharmaceutically acceptable salts thereof.

13. A method of inhibiting one or more inflammatory processes selected from the group consisting of: proinflammatory cytokine production, lymphocyte proliferation, granulocyte degranulation, t-cell proliferation, neutrophilia, and oedema comprising exposing an organ or tissue afflicted with inflammation to an amount of a compound of Formula (I) effective to inhibit said inflammatory process.

14. A method for inhibiting pro-inflammatory cytokine production comprising exposing human peripheral leukocytes to an amount of a compound of Formula (I) effective to reduce production of at least one of TNF-α, IL-1, IL-6, IL-8, IL-2 or IL-5 compared to control leukocytes.

15. A method for inhibiting human T-cell proliferation comprising exposing human T-cells to an amount of a compound of Formula (I) effective to reduce production of said T-cells compared to control T cells not exposed to said compound.

16. The method of claim 12, wherein the inflammatory process comprises proinflammatory cytokine production, comprising exposing human peripheral leukocytes to an amount of a compound of Formula (I) effective to reduce production of at least one of TNF-α, IL-1, IL-6, IL-8, IL-2 or IL-5 compared to control leukocytes.

17. The method of claim 15, wherein the production of TNF-α is reduced.

18. The method of claim 15, wherein the production of IL-1α and/or IL-1β is reduced.

19. The method of claim 15, wherein the production of IL-2 and/or IL-5 is reduced.

20. The method of claim 12, wherein the inflammatory process comprises T-cell proliferation comprising exposing human T-cells to an amount of a compound according to claim 1 effective to reduce production of said T-cells compared to control T cells not exposed to said compound.

21. The method of claim 12, wherein the inflammatory process comprises granulocyte degranulation.

22. The method of claim 12, wherein the inflammatory process comprises granulocyte degranulation, comprising exposing human granulocytes to an amount of a compound according to claim I effective to reduce granulocyte degranulation.

23. The method of claim 12, wherein the inflammatory process comprises lymphocyte proliferation.

24. The method of claim 12, wherein the immune response to an antigen is inhibited.

25. The method of claim 12, wherein the inflammatory process comprises neutrophilia.

26. The method of claim 12, wherein the inflammatory process comprises oedema.

27. The method of claim 12, wherein the inhibition of the inflammatory process comprises inhibiting the production of cytokines, the production of T-cells, the degranulation of granulocytes, cell growth, or neutrophil production, by at least 50%.

28. The method of claim 26, wherein the inhibition is at least 90%.

29. The method of claim 9, wherein the compound of formula I is selected from the group consisting of: 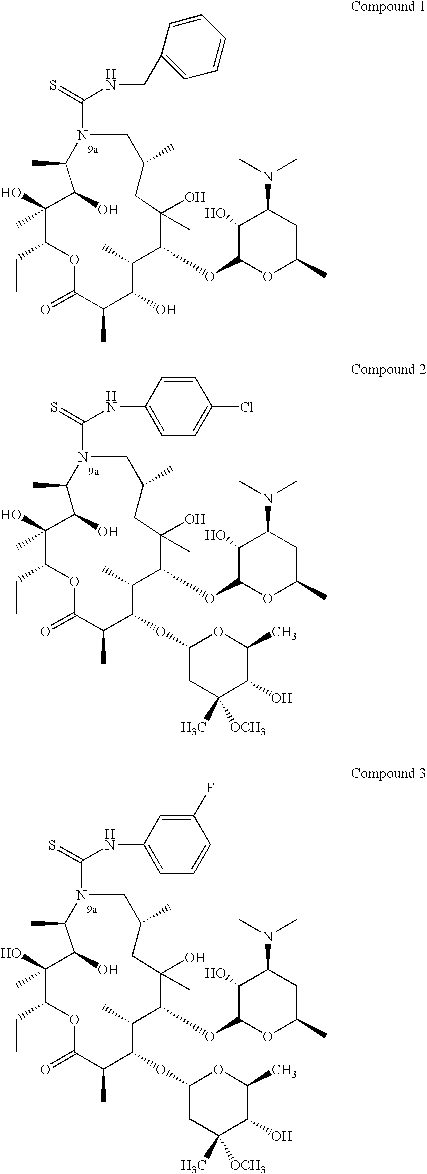 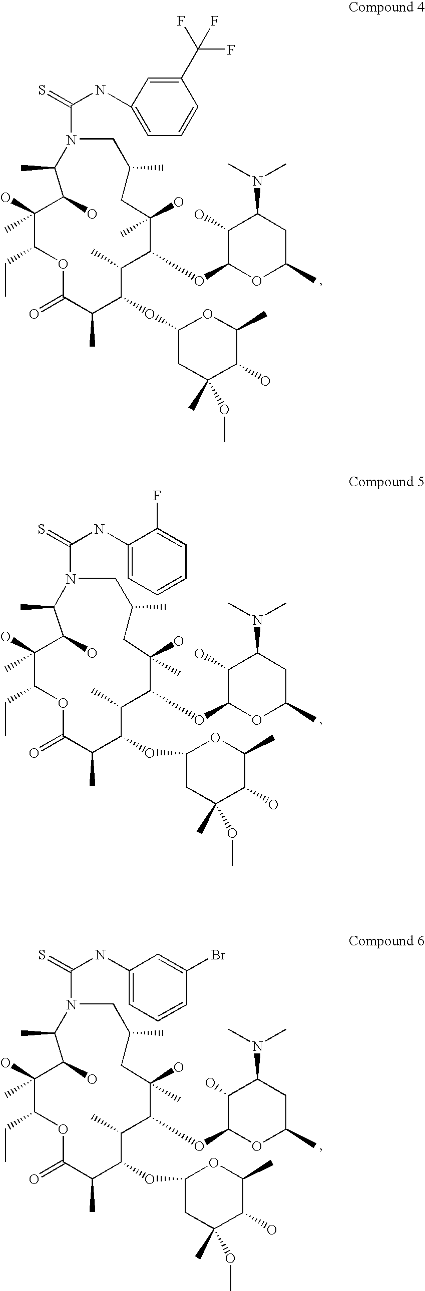 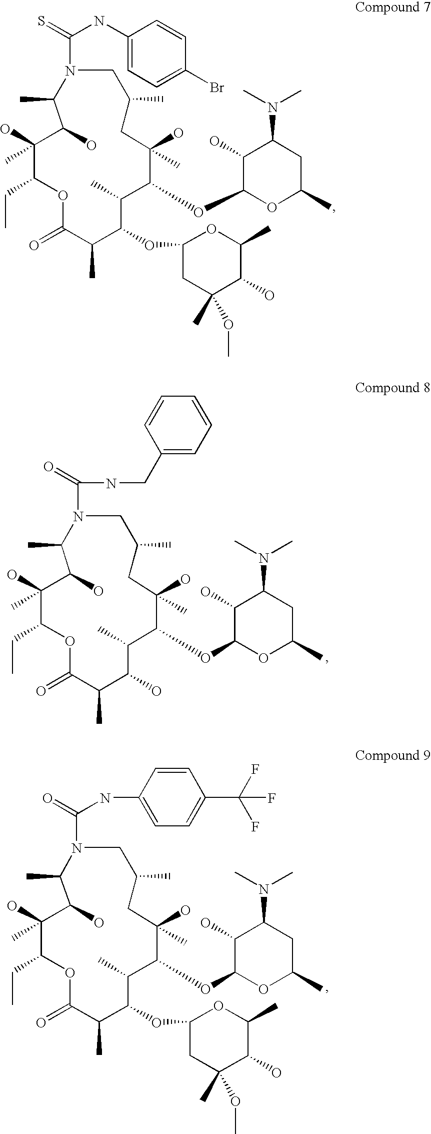 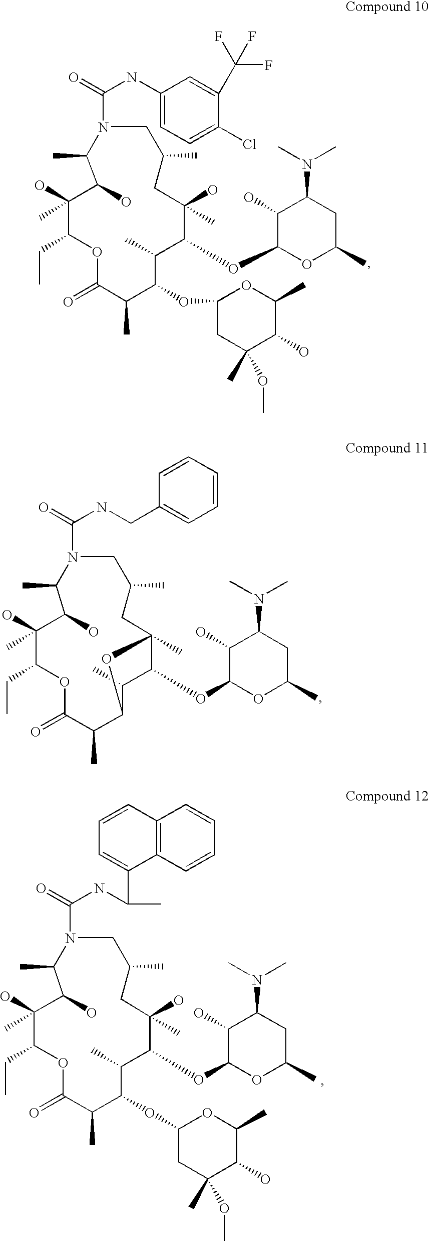 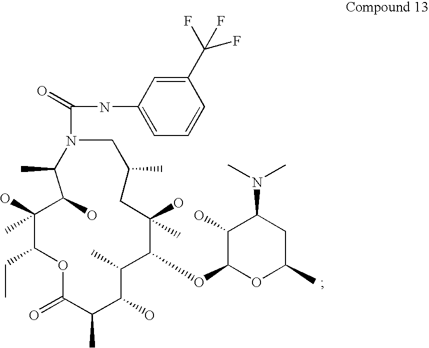
and pharmaceutically acceptable salts thereof.
